(19) United States
(12) Patent Application Publication
(10) Pub. No.: US 2026/0027027 A1
(43) Pub. Date: Jan. 29, 2026

(54) FRAGRANCED COMPOSITION COMPRISING AT LEAST ONE SULFUR-COMPRISING ANTIOXIDANT COMPOUND, AT LEAST ONE ORGANIC UV SCREENING AGENT AND AT LEAST ONE FRAGRANCING SUBSTANCE

(71) Applicant: L'OREAL, PARIS (FR)

(72) Inventor: Christophe BOBIN, CHEVILLY LARUE (FR)

(21) Appl. No.: 18/995,693

(22) PCT Filed: Jul. 20, 2023
(86) PCT No.: PCT/EP2023/070197

§ 371 Date: Jan. 16, 2025

(30) Foreign Application Priority Data
Jul. 21, 2022 (FR) FR2207506

(51) Int. Cl. A61K 8/46; A61K 8/35; A61Q 13/00; A61Q 17/04

(52) U.S. Cl. CPC A61K 8/46; A61K 8/35; A61Q 13/00; A61Q 17/04; A61K 2800/522

(57) ABSTRACT

The present invention relates to a fragranced composition, in particular cosmetic composition, comprising a) at least one sulfur-comprising antioxidant compound chosen from the compounds of formulae (I) R₁—S—R₂ and/or (II) R₁—S—S—R₂, b) at least one organic UV screening agent chosen from dibenzoylmethane derivatives; and c) at least one fragrancing substance.

### TECHNICAL FIELD

The present invention is targeted at providing a novel fragranced composition, in particular cosmetic composition, for the field of the fragrancing of keratin materials and/or clothing, but also of the care and/or cosmetic treatment of keratin materials.

### PRIOR ART

In general, the formulation of environmentally friendly cosmetic products is becoming a major challenge for meeting new consumer expectations, in particular regarding natural and/or eco-friendly products, that is to say products, the design and development of which take into account environmental impacts.

Formulations of cosmetic products which make it possible to protect the consumer, in particular while avoiding the use of compounds which may be the cause of suspicion on the part of the consumer, for example those suspected of being an endocrine disruptor, are more particularly desired.

These environmental problems concern all cosmetic products, notably fragranced compositions.

It is known that a fragrance is a combination of different odoriferous substances which evaporate at different periods. Each fragrance exhibits what is known as a “top note”, which is the odour which diffuses first during the application of the fragrance or when the receptacle containing it is opened, a “middle or heart note”, which corresponds to the full fragrance (given off for a few hours after the “top note”), and a “base note”, which is the most persistent odour (given off for several hours after the “middle note”). The persistence of the base note corresponds to the holding of the fragrance.

Human beings have always sought to fragrance themselves and to fragrance the objects surrounding them or their environments, both to mask strong and/or unpleasant odours and to give a nice odour.

It is common practice to incorporate fragrance into a certain number of products or compositions, in particular cosmetic and dermatological compositions, such as eaux fraîches, eaux de toilette, eaux de parfum, aftershave lotions or eaux de soin.

A fragrance or a fragrancing composition has to be stable, in particular olfactorily stable, and to have a pleasant odour, that is to say that they must not degrade olfactorily over time.

They must be resistant to different attacking factors, such as light and temperature differences. There is a great advantage in addition in managing to stabilize the colour of a fragrance or of a fragrancing composition.

In order to ensure the stability of fragranced formulations, and in particular to prevent a change in odour, it is known to incorporate an antioxidant system, such as BHT, therein.

However, BHT is a controversial compound, as it is supposedly toxic to human beings. In addition, it is suspected of being environmentally unfriendly and of not being biodegradable.

In point of fact, to the knowledge of the inventors, no substitutes for BHT for fragranced aqueous/alcoholic products are known to date.

The search for alternative antioxidant systems to BHT is thus a major line of study.

### DISCLOSURE OF THE INVENTION

A need thus remains to look for novel fragranced compositions, in particular aqueous/alcoholic compositions, which remain stable over time, under the effects of light and temperature.

In particular, a need remains for novel fragranced compositions, in particular aqueous/alcoholic compositions, which have an olfactory stability over time and under the effects of light and temperature.

In particular, a need remains to have available such fragranced cosmetic compositions, the change in the organoleptic properties of which, namely the odour and/or the colour, in particular at least the colour, is controlled over time.

A need remains in particular to have available fragranced compositions which make it possible to retain the olfactory outcome over time, without loss of strength.

In particular, a need remains for additives which make it possible to olfactorily stabilize the majority of the olfactory families of perfumery, indeed all the olfactory families.

There also remains a need to increase the persistence of such fragrancing compositions on keratin materials without the fragrance degrading, in particular on contact with the other constituents of the composition.

In addition, a need remains to have available olfactorily stable formulations which can observe industrial constraints and in particular which can be easily manufactured.

Finally, there also remains a need to have available fragrancing compositions which are compatible with current consumer demands, in particular from an environmental viewpoint.

The present invention is specifically targeted at meeting these needs.

### SUMMARY OF THE INVENTION

Thus, according to a first of its aspects, the present invention relates to a fragranced composition, in particular cosmetic composition, comprising:
a) at least one sulfur-comprising antioxidant compound chosen from the compounds of following formulae (I) and/or (II), and also their optical and geometrical isomers, salts and solvates, such as hydrates:

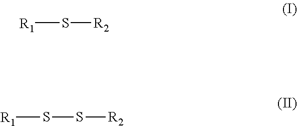

in which formulae (I) and (II):
   R₁ and R₂, which are identical or different, represent a linear or branched (C₁-C₂₄)alkyl radical, a linear or branched (C₂-C₁₈)alkenyl radical, a (C₅-C₁₂)cycloalkyl radical, a phenylalkyl radical having from 7 to 15 carbon atoms or a phenyl radical,
said alkyl radical and/or said alkenyl radical optionally being substituted by one or more group(s) chosen from the —OH, —O—C(O)—R₃, —C(O)—O—R₃, —C(O)—R₃, —OR′₃ and —NH₂ groups, in particular —OH, —OR′₃ and —NH₂ groups, and/or
said alkyl radical and/or said alkenyl radical optionally being interrupted by one or more group(s) chosen from heteroatoms, such as —O—, or —N(H)—, —O—C(O)—, —C(O)—O—, —C(O)— and —NR′₃— groups,
said phenyl radical and/or said phenylalkyl radical, such as benzyl, optionally being substituted on the phenyl by 1 to 3 linear or branched (C₁-C₁₀)alkyl radicals,
with R₃ independently representing a hydrogen atom, a linear or branched (C₁-C₁₈)alkyl radical, a (C₅-C₁₂)cycloalkyl radical, a linear or branched (C₃-C₈)alkenyl radical, a (C₆-C₁₄)aryl radical or a (C₇-C₁₅)aralkyl radical, and
   R′₃ representing a linear or branched (C₁-C₁₈)alkyl radical;
b) at least one organic UV screening agent chosen from dibenzoylmethane derivatives; and
c) at least one fragrancing substance.

Preferably, in a composition according to the invention, the sulfur-comprising antioxidant compound a) is chosen from dilauryl thiodipropionate, distearyl thiodipropionate, dilauryl dithiodipropionate, distearyl dithiodipropionate and their mixtures, preferably from dilauryl thiodipropionate, distearyl thiodipropionate and their mixture.

Preferably, the composition according to the invention comprises at least dilauryl thiodipropionate in combination with butyl methoxydibenzoylmethane.

The inventors have found, surprisingly, that the combination of a specific sulfur-comprising antioxidant compound and of an organic UV screening agent derived from dibenzoylmethane, in the presence of fragrancing substances, makes it possible to obtain a fragranced cosmetic composition having an excellent olfactory stability, similar to, indeed even superior to, that obtained by use of BHT, and the odour and the olfactory power of which degrades only slightly over time.

Specifically, as emerges from the examples appearing below, the compositions according to the invention are stable, in the sense that the change in the odour and in its power remain low over time, even under conditions simulating an accelerated ageing of the composition.

It is also noted that the compositions according to the invention are stable in terms of intensity of the colour, that is to say that the intensity of the colour decreases slightly over time.

Moreover, it is observed that the specific combination employed in the composition according to the invention additionally makes it possible to obtain excellent performance qualities in terms of olfactory stability for numerous olfactory families of perfumery.

In particular, the use of the sulfur-comprising antioxidant compound with an organic UV screening agent derived from dibenzoylmethane makes it possible to formulate aqueous/alcoholic fragrancing compositions with a lifetime of at least 3 years, indeed even ranging up to 5 years.

A composition according to the invention is intended in particular to be applied to keratin materials or clothing.

Thus, the invention relates, according to another of its aspects, to a method for the cosmetic treatment of keratin materials, in particular of the skin, comprising at least a stage of application, to said keratin materials, of a composition according to the invention.

The invention also relates to a method for the cosmetic treatment of keratin materials, in particular of the skin, or of clothing, comprising at least a stage of application, to said keratin materials and/or clothing, of a fragranced composition according to the invention.

It is understood that the cosmetic treatment methods targeted in the present patent application are non-therapeutic.

A composition according to the invention is intended in particular to be employed in order to fragrance keratin materials and/or clothes.

Thus, the invention also relates, according to another of its aspects, to a method for fragrancing keratin materials, and in particular the skin, and/or clothing, preferably the skin, comprising the application, to said keratin materials and/or said clothing, of the composition as defined above.

Advantageously, the composition can be applied by spraying, in particular using a sprayer.

According to yet another of its aspects, the invention relates to a use of at least one sulfur-comprising antioxidant compound, in particular as described in the present text, chosen from the compounds of following formulae (I) and/or (II), and also their optical and geometrical isomers, salts and solvates, such as hydrates:

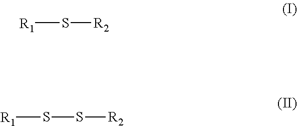

in which formulae (I) and (II):
R₁ and R₂, which are identical or different, represent a linear or branched (C₁-C₂₄)alkyl radical, a linear or branched (C₂-C₁₈)alkenyl radical, a (C₅-C₁₂)cycloalkyl radical, a phenylalkyl radical having from 7 to 15 carbon atoms or a phenyl radical,
said alkyl radical and/or said alkenyl radical optionally being substituted by one or more group(s) chosen from the —OH, —O—C(O)—R₃, —C(O)—O—R₃, —C(O)—R₃, —O—R′₃ and —NH₂ groups, in particular —OH, —OR′₃ and —NH₂ groups, and/or
said alkyl radical and/or said alkenyl radical optionally being interrupted by one or more group(s) chosen from heteroatoms, such as —O—, or —N(H)—, —O—C(O)—, —C(O)—O—, —C(O)— or —NR′₃— groups,
said phenyl radical and/or said phenylalkyl radical, such as benzyl, optionally being substituted on the phenyl by 1 to 3 linear or branched (C₁-C₁₀)alkyl radicals,
with R₃ independently representing a hydrogen atom, a linear or branched (C₁-C₁₈)alkyl radical, a (C₅-C₁₂)cycloalkyl radical, a linear or branched (C₃-C₈)alkenyl radical, a (C₆-C₁₄)aryl radical or a (C₇-C₁₅)aralkyl radical, and
R′₃ representing a linear or branched (C₁-C₁₈)alkyl radical;
as stabilizer in an in particular aqueous or aqueous/alcoholic, in particular cosmetic, fragranced composition additionally comprising at least one organic UV screening agent chosen from dibenzoylmethane derivatives, in particular as described in the present text, and at least one fragrancing substance, in particular as described in the present text.

In particular, the use according to the invention employs the sulfur-comprising antioxidant compound, the organic UV screening agent and the fragrancing substance in the form of a composition according to the invention.

### Definitions

In the context of the present invention, and unless otherwise indicated, the following definitions apply:

Within the meaning of the present invention, the term “fragranced composition” or “fragrancing composition” or “fragrance” is understood to denote any composition which leaves a fragrance on keratin materials after application.

Within the meaning of the present invention, the term “fragrancing substance” is understood to denote any fragrance, odorous starting material or aroma which is capable of giving off a pleasant odour, in particular as defined in the continuation of the text. Within the meaning of the present invention, “fragrancing”, “odorous” or “odoriferous” substances are synonymous.

Within the meaning of the present invention, the term “natural” is understood to denote a compound or extract obtained directly from the earth or the soil, or from plants or animals, via, if appropriate, one or more physical processes, such as, for example, milling, refining, distillation, purification or filtration, or also resulting from a biotechnological process, in particular resulting from microbiological or cell cultures, for example from fungi or from bacteria. “Natural” compounds include compounds which are present in nature and which can be reproduced by chemical (semi)synthesis.

Within the meaning of the present invention, the term “of natural origin” is understood to denote any compound obtained from a natural substance which has undergone one or more additional chemical or industrial treatments, bringing about modifications which do not affect the essential qualities of this substance. Mention may be made, as non-limiting example of additional chemical or industrial treatment bringing about modifications which do not affect the essential qualities of a natural compound, of those authorized by the controlling bodies, such as Ecocert (Reference system for biological and ecological cosmetic products, January 2003), or defined in recognized handbooks in the field, such as “Cosmetics and Toiletries Magazine”, 2005, Vol. 120, 9 (10).

According to the invention, a compound is considered to be natural or of natural origin when it is predominantly composed of natural constituents, that is to say when the ratio by weight of natural constituents to non-natural constituents which make up the compound is greater than 1.

Within the meaning of the present invention, the term “keratin materials” is understood in particular to denote the skin, the lips, the hair, the scalp, the eyelashes and the eyebrows or also the nails, in particular the skin and/or the lips, and preferably the skin.

The term “dye” or “colorant” is understood to denote any compound capable of colouring the fragranced composition, that is to say any compound which absorbs in the visible spectrum, in particular so as to appear to the human eye to have a colour such as yellow, orange, red, purple, blue or green.

A composition according to the invention is generally suitable for application to keratin materials, in particular topical application to the skin, and thus generally comprises a physiologically acceptable medium, that is to say a medium compatible with the skin.

It is preferably a cosmetically acceptable medium, that is to say a medium which exhibits a pleasant colour, odour and feel and which does not cause any unacceptable discomfort, that is to say stinging, tautness or redness, liable to dissuade the user from applying this composition.

Within the meaning of the present invention, a “stabilizer” is understood to denote a compound which is capable of stabilizing the fragranced composition comprising it, in particular in terms of preservation of its organoleptic properties with respect to external attacking factors, in particular light, or temperature or oxygen differences, in particular the colour and/or the odour of said composition.

A (Cₓ-C_{z})alkyl group represents a linear or branched hydrocarbon chain comprising from x to z carbon atoms. For example, a (C₁-C₆)alkyl group represents a linear or branched hydrocarbon chain comprising from 1 to 6 carbon atoms.

A (Cₓ-C_{z})alkenyl group represents a linear or branched hydrocarbon chain comprising from x to z carbon atoms, and comprising one or more conjugated or non-conjugated unsaturations, preferably only one unsaturation. For example, a (C₂-C₁₀)alkenyl group represents a linear or branched hydrocarbon chain comprising from 2 to 10 carbon atoms and comprising one or more unsaturations.

A (Cₓ-C_{z})alkoxy group represents an —O—(Cₓ-C_{z})alkyl radical in which the (Cₓ-C_{z})alkyl group is as defined above.

A (Cₓ-C_{z})cycloalkyl group represents a non-aromatic, saturated or unsaturated, monocyclic or fused or non-fused polycyclic, carbocycle (cyclic hydrocarbon group) comprising from x to z carbon atoms.

A (Cₓ-C_{z})aryl group represents a monocyclic or fused or non-fused polycyclic carbocycle comprising from x to z carbon atoms, and at least one ring of which is aromatic; preferentially, the aryl radical is a phenyl, biphenyl, naphthyl, indenyl, anthracenyl or tetrahydronaphthyl, preferably phenyl.

A (Cₓ-C_{z})aralkyl group represents an alkyl radical substituted by an aryl radical, comprising from x to z carbon atoms.

The expression “at least one” is equivalent to “one or more”.

The expressions “of between . . . and . . . ”, “comprises from . . . to . . . ”, “formed from . . . to . . . ” and “ranging from . . . to . . . ” should be understood as being inclusive of the limits, unless otherwise specified.

Other characteristics, alternative forms and advantages of the compositions according to the invention will emerge more clearly on reading the description and the examples which will follow.

### DETAILED DESCRIPTION

### a) Sulfur-Comprising Antioxidant Compound

As mentioned above, a fragranced composition according to the invention comprises at least one sulfur-comprising antioxidant compound chosen from the compounds of following formulae (I) and/or (II), and also their optical and geometrical isomers, salts and solvates, such as hydrates:

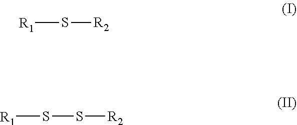

in which formulae (I) and (II):
R₁ and R₂, which are identical or different, represent a linear or branched (C₁-C₂₄)alkyl radical, a linear or branched (C₂-C₁₈)alkenyl radical, a (C₅-C₁₂)cycloalkyl radical, a phenylalkyl radical having from 7 to 15 carbon atoms or a phenyl radical,
said alkyl radical and/or said alkenyl radical optionally being substituted by one or more group(s) chosen from the —OH, —O—C(O)—R₃, —C(O)—O—R₃, —C(O)—R₃, —OR′₃ and —NH₂ groups, in particular —OH, —OR′₃ and —NH₂ groups, and/or
said alkyl radical and/or said alkenyl radical optionally being interrupted by one or more group(s) chosen from heteroatoms, such as —O—, or —N(H)—, —O—C(O)—, —C(O)—O—, —C(O)— or —NR′₃— groups,
said phenyl radical and/or said phenylalkyl radical, such as benzyl, optionally being substituted on the phenyl by 1 to 3 linear or branched (C₁-C₁₀)alkyl radicals,
with R₃ independently representing a hydrogen atom, a linear or branched (C₁-C₁₈)alkyl radical, a (C₅-C₁₂)cycloalkyl radical, a linear or branched (C₃-C₈)alkenyl radical, a (C₆-C₁₄)aryl radical or a (C₇-C₁₅)aralkyl radical, and
R′₃ representing a linear or branched (C₁-C₁₈)alkyl radical.

Preferably, R₁ and R₂ are identical.

In particular, R₁ and R₂, which are identical or different, represent a linear or branched (C₁-C₂₄)alkyl, in particular (C₅-C₂₀)alkyl, indeed even (C₁₂-C₁₆)alkyl, radical interrupted by one or more —O—C(O)— or —C(O)—O— group(s) and being optionally substituted by one or more group(s) chosen from the —OH, —O—C(O)—R₃, —C(O)—O—R₃, —C(O)—R₃, —O—R′₃ and —NH₂ groups, in particular —OH, —O—R′₃ and —NH₂ groups, more particularly —OH and —NH₂ groups, preferably being unsubstituted.

In particular, the sulfur-comprising antioxidant compound(s) are chosen from the mercapto (or thio) compounds of formula (I) as are defined above. More particularly, the mercapto compounds of formula (I) are chosen from which R₁ and R₂ are identical and represent a group chosen from i) R′₃—O—C(O)-ALK- and ii) R′₃—C(O)—O-ALK-, preferably i) R′₃—O—C(O)-ALK-, with ALK representing a linear or branched (C₁-C₆)alkylene group, such as methylene or ethylene, preferably ethylene, and R′₃ being as defined above, in particular R′₃ representing a linear or branched (C₆-C₁₆)alkyl group; more particularly, R′₃ represents a linear or branched (C₅-C₁₄)alkyl group, preferentially a linear or branched (C₁₀-C₁₂)alkyl group, more preferentially R′₃ is a linear alkyl group, such as n-dodecyl (C₁₂H₂₅).

In particular, the sulfur-comprising antioxidant compound is chosen from the diesters of thiodipropanoic acid, the diesters of dithiodipropanoic acid and their mixture.

Preferably, the fragranced composition comprises at least one sulfur-comprising antioxidant compound a) chosen from dilauryl thiodipropionate, distearyl thiodipropionate, dilauryl dithiodipropionate, distearyl dithiodipropionate and their mixtures, preferably from dilauryl thiodipropionate, for example that sold under the trade name Tinogard® DA by BASF, distearyl thiodipropionate and their mixture.

According to a preferred embodiment, the fragranced composition comprises at least dilauryl thiodipropionate.

According to another specific embodiment, said sulfur-comprising antioxidant compound a) is chosen from the compounds of formula (I) having carboxylic acid group(s), in particular those for which R₁ and R₂ are identical and represent an -ALK-C(O)—OH group with ALK as defined above, in particular methylene or ethylene, preferably ethylene, and their derivatives.

According to another specific embodiment, said sulfur-comprising antioxidant compound a) is chosen from thiodipropanoic acid, dithiodipropanoic acid, thiodipropanoic acid derivatives, dithiodipropanoic acid derivatives and their mixtures.

Mention may be made, as thiodipropanoic acid and dithiodipropanoic acid derivatives, of monoesters, diesters and salts of thiodipropanoic acid and monoesters, diesters and salts of dithiodipropanoic acid.

The esters of thiodipropanoic acid and the esters of dithiodipropanoic acid can be monoesters or diesters of thiodipropanoic acid and of alcohol(s) R—OH, or monoesters or diesters of dithiodipropanoic acid and of alcohol(s) R—OH, avec R being a linear or branched, preferably linear, (C₄-C₂₀)alkyl, in particular (C₆-C₁₆)alkyl, indeed even (C₅-C₁₄)alkyl, radical, preferentially a linear or branched (C₁₀-C₁₂)alkyl radical; more preferentially, R is a linear alkyl group, such as n-dodecyl (C₁H₂₅). R can also be a (C₁₂-C₁₈)alkyl radical.

The composition can comprise at least 0.01% by weight, in particular at least 0.05% by weight, indeed even at least 0.08% by weight, indeed even again at least 0.1% by weight, of sulfur-comprising antioxidant compound(s) a), in particular of dilauryl thiodipropionate, with respect to the total weight of said fragranced composition.

The composition can comprise at most 4% by weight, in particular at most 1% by weight, indeed even at most 0.1% by weight, of sulfur-comprising antioxidant compound(s) a), with respect to the total weight of said fragranced composition.

Preferably, the composition comprises from 0.01% to 5% by weight, in particular from 0.03% to 2% by weight, especially from 0.05% to 1% by weight, preferably from 0.07% to 0.5% by weight, more preferentially from 0.08% to 0.1% by weight, of sulfur-comprising antioxidant compound(s) a), in particular of dilauryl thiodipropionate, with respect to the total weight of said fragranced composition.

In particular, the sulfur-comprising antioxidant compound(s) a) are employed in a sulfur-comprising antioxidant compound(s) a)/fragrancing substance(s) c) ratio by weight ranging from 0.05/100 to 2/100, in particular from 0.1/100 to 0.9/100, more particularly from 0.3/100 to 0.7/100.

In particular, the sulfur-comprising antioxidant compound(s) a) and the organic UV screening agent(s) b) are employed in a sulfur-comprising antioxidant compound(s) a)/organic UV screening agent(s) b) ratio by weight of less than or equal to 2, more particularly of less than or equal to 1, preferably of less than 1, better still from 0.01 to 0.9, more particularly from 0.05 to 0.6, preferentially from 0.1 to 0.5, such as 0.3.

### Organic UV Screening Agents

As mentioned above, a fragranced composition according to the invention also comprises at least one organic UV screening agent chosen from dibenzoylmethane derivatives.

Within the meaning of the present invention, the term “UV screening agent” is understood to denote any compound which screens out ultraviolet (UV) radiation in the wavelength range extending from 280 nm to 400 nm.

In particular, the dibenzoylmethane derivatives can be compounds corresponding to the formula (III) below and also their optical and geometrical isomers, and also their organic or inorganic acid or base salts, and their solvates;

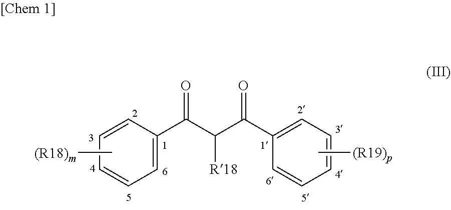

in which:
   R18, which is identical or different, represents a group chosen from those of following formulae (A) to (H):

TABLE 1

| | |
|---|---|
| 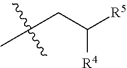 | (A) |
| 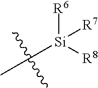 | (B) |
| 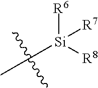 | (C) |
| 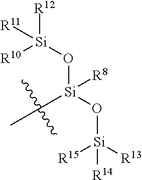 | (D) |
| 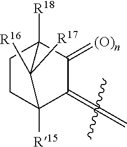 | (E) |
| 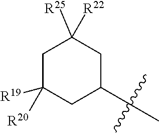 | (F) |
| 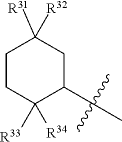 | (F⁺) |
| 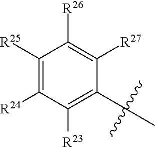 | (G) |
| 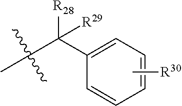 | (H) |

in which:
   R⁴ and R⁵, which are identical or different, preferably different, represent a (C₂-C₁₀)alkyl, in particular (C₂-C₅)alkyl, more particularly (C₂-C₆)alkyl, group; preferably, R⁴ represents an ethyl group and R⁵ represents an n-butyl group;
R⁶, R⁷ and R⁸, which are identical or different, preferably identical, represent a (C₁-C₆)alkyl group, a phenyl or benzyl group, preferably a (C₁-C₄)alkyl group, such as methyl or ethyl, or else R⁶ and R⁷ are identical and represent a (C₁-C₄)alkyl group, such as methyl, and R⁸, which is different from R⁶ and R⁷, represents a (C₁-C₆)alkyl group, preferably a branched (C₄-C₆)alkyl group, such as 2,2-dimethylpropyl;
R′⁶, R′⁷ and R′⁸, which are identical or different, preferably identical, represent a (C₁-C₆)alkyl group, a (C₁-C₆)alkoxy group, or a phenyl, benzyl, phenoxy or benzoxy group, preferably a (C₁-C₄)alkyl group and in particular a methyl group;
R⁹ represents a (C₁-C₆)alkyl group, preferably a (C₁-C₄)alkyl group, in particular a methyl group;
R¹⁰, R¹¹, R¹², R¹³, R¹⁴ and R¹⁵, which are identical or different, preferably identical, represent a (C₁-C₆)alkyl group, preferably a (C₁-C₄)alkyl group, in particular a methyl group;
R¹⁶, R¹⁷ and R¹⁸, which are identical or different, preferably identical, represent a hydrogen atom, a (C₁-C₆)alkyl group, preferably a (C₁-C₄)alkyl group, in particular a methyl group;
R¹⁸ represents a hydrogen atom or a (C₁-C₆)alkyl group, preferably a hydrogen atom or a methyl group;
n has the value 0 or 1, preferably 1;
R¹⁹, R²⁰, R²¹ and R²², which are identical or different, preferably identical, represent a (C₁-C₆)alkyl group, a (C₁-C₆)alkoxy group, preferably a (C₁-C₄)alkyl group, in particular a methyl group, it being understood that two alkoxy radicals cannot be carried by the same carbon atom;
R²³, R²⁴, R²⁵, R²⁶ and R²⁷, which are identical or different, represent a hydrogen atom or a (C₁-C₆)alkyl group, it being understood that at least one of the R²³, R²⁴, R²⁵, R²⁶ and R²⁷ groups represents a t-butyl group; preferably, R²³, R²⁴, R²⁶ and R²⁷ represent a hydrogen atom and R²⁵ represents a t-butyl group;
R²⁸ and R²⁹, which are identical or different, preferably identical, represent a (C₁-C₆)alkyl group, preferably a (C₁-C₄)alkyl group, in particular a methyl group;
R³⁰ represents a hydrogen atom or a (C₁-C₆)alkyl group, particularly a (C₁-C₄)alkyl group, in particular a methyl group; preferably, R³⁰ represents a hydrogen atom;
R³¹ and R³², which are identical or different, preferably identical, represent a hydrogen atom or a (C₁-C₆)alkyl group, in particular a hydrogen atom;
R³³ and R³⁴, which are identical or different, preferably different, represent a (C₁-C₆)alkyl group, preferably a (C₁-C₄)alkyl group, such as a methyl, ethyl, propyl or isopropyl group;
the unit:

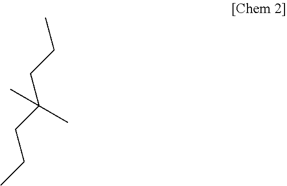

represents the part of the group which is connected to the remainder of the molecule; and
the unit:

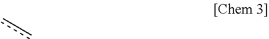

represents a single or double bond, preferably a double bond;
or R18, which is identical or different, represents a hydrogen atom or a linear or branched C₁-C₈ alkyl group, preferably a branched C₄-C₈ alkyl group; more particularly, R18 represents a group of formula (B) as defined above, or a linear or branched C₁ to C₆ alkyl group;
R′18 represent a hydrogen atom or a linear or branched, particularly branched, (C₁-C₆)alkyl group, such as an isopropyl group;
R19, which is identical or different, represents a hydrogen atom, a linear or branched C₁ to C₆ alkyl group or a linear or branched C₁ to C₄ alkoxy group, in particular a (C₁-C₄)alkyl group, such as a methyl, ethyl, propyl or isopropyl group, a methoxy or ethoxy group;
m represents an integer of between 1 and 5 inclusive, in particular has the value 1 or 2; preferably 1; more particularly, R18 is found in the 4 position;
p represents an integer of between 1 and 5 inclusive, in particular has the value 1 or 2; preferably 1; more particularly, R19 is found in the 4′ position;
   it being understood that R18, R′18 and R19 cannot simultaneously represent a hydrogen atom.

In particular, the dibenzoylmethane derivatives can be chosen from the compounds of formula (III) in which R18 and R19, which are identical or different, preferably different, represent a group chosen from (A) to (H) as defined above, in particular linear or branched C₁ to C₁₆ alkyl; or a linear or branched C₁ to C₁₆ alkoxy group, in particular a (C₁-C₈)alkoxy group; or a hydrogen atom; preferably, R19 represents a C₁-C₆, preferably C₁-C₄, alkoxy group, such as methoxy, and R18 represents a group chosen from (A) to (H), and in particular a linear or branched C₁-C₈ alkyl group, preferably a branched C₄-C₈ alkyl group, and more particularly R18 represents a group chosen from the groups of formula (B), in particular a tert-butyl group, with in particular R′18 representing a hydrogen atom, m and p having the value 1, and R18 and R19 occurring respectively in the 4 and 4′ positions.

In particular, the dibenzoylmethane derivatives can be chosen from the compounds of formula (III) in which:
R18 represents a hydrogen atom or a linear or branched C₁ to C₆ alkyl group, preferably a hydrogen atom; and
R19 represents a linear or branched C₁ to C₆ alkyl group or a linear or branched C₁ to C₄ alkoxy group, in particular a (C₁-C₈)alkyl group, such as a methyl, ethyl, propyl or isopropyl group;
   with in particular R′18 representing a hydrogen atom, m and p having the value 1, and R18 and R19 occurring respectively in the 4 and 4′ positions.

In particular, the dibenzoylmethane derivatives can be chosen from 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 4-tert-butyl-4′-methoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane and 2,6-dimethyl-4-tert-butyl-4′-methoxydibenzoylmethane.

Preferably, the dibenzoylmethane derivatives are chosen from Butyl Methoxydibenzoylmethane, also called t-Butyl Methoxydibenzoylmethane or also 4-tert-butyl-4′-methoxydibenzoylmethane, for example that sold under the trade name Parsol 1789 by Hoffmann-La Roche, and Isopropyl Dibenzoylmethane, also called 4-isopropyldibenzoylmethane, for example that sold under the trade name Eusolex 8020 by Merck, and their mixture.

Preferably, the fragranced composition comprises at least Butyl Methoxydibenzoylmethane.

According to a specific embodiment, the composition can comprise at least 0.05% by weight, in particular at least 0.1% by weight, more particularly at least 0.2% by weight, indeed even at least 0.25% by weight, of organic UV screening agent(s) b) chosen from dibenzoylmethane derivatives, in particular Butyl Methoxydibenzoylmethane, with respect to the total weight of said fragranced composition.

Preferably, the composition comprises from 0.01% to 5% by weight, in particular from 0.05% to 2% by weight, especially from 0.1% to 1% by weight, preferably from 0.2% to 0.5% by weight, more preferentially from 0.25% to 0.35% by weight, such as 0.3% by weight, of organic UV screening agent(s) b) chosen from dibenzoylmethane derivatives, in particular of Butyl Methoxydibenzoylmethane, with respect to the total weight of said fragranced composition.

In particular, the organic UV screening agent(s) b) are employed in an organic UV screening agent(s) b)/fragrancing substance(s) c) ratio by weight ranging from 0.1/100 to 5/100, in particular from 0.5/100 to 3/100, more particularly from 1/100 to 2/100.

### c) Fragrancing Substance(s)

As mentioned above, a composition according to the invention comprises at least one fragrancing substance.

Fragrances are compositions containing in particular the starting materials described in S. Arctander, Perfume and Flavor Chemicals (Montclair, N.J., 1969), in S. Arctander, Perfume and Flavor Materials of Natural Origin (Elizabeth, N.J., 1960), and in Flavor and Fragrance Materials —1991, Allured Publishing Co., Wheaton, Ill.

A fragranced composition according to the invention preferably comprises at least one fragrancing substance chosen from essential oils, fragrances and aromas of synthetic or natural origin, and their mixtures.

They can be natural products, such as essential oils, absolutes, resinoids, resins, concretes and/or synthetic products, such as terpene or sesquiterpene hydrocarbons, alcohols, phenols, aldehydes, ketones, ethers, acids, esters, nitriles or peroxides, which are saturated or unsaturated, aliphatic or cyclic.

According to the definition given in International Standard ISO 9235 and adopted by the European Pharmacopoeia Commission, an essential oil is an odorous product, generally of complex composition, obtained from a botanically defined plant starting material, either by steam distillation, or by dry distillation, or by an appropriate mechanical process without heating (cold expression). The essential oil is generally separated from the aqueous phase by a physical process which does not result in any significant change in the composition.

The choice of the method for obtaining the essential oils depends mainly on the starting material: its original state and its characteristics, its intrinsic nature. The “essential oil/plant starting material” yield may be extremely variable depending on the plant: 15 ppm to more than 20%. This choice determines the characteristics of the essential oil, in particular viscosity, colour, solubility, volatility, and enrichment or depletion in certain constituents.

Steam distillation corresponds to the vaporization, in the presence of steam, of a sparingly water-miscible substance. The starting material is placed in contact with boiling water or steam in a still. The steam entrains the essential oil vapour, which is condensed in the condenser in order to be recovered as liquid phase in a Florentine flask (or essence jar), where the essential oil is separated from the water by settling. The term “aromatic water” or “hydrolat” or “distilled floral water” is used to describe the aqueous distillate which remains after the steam distillation, once the essential oil has been separated.

Production by dry distillation consists in obtaining the essential oil by distillation of wood, bark or roots, without addition of water or steam, in a closed chamber designed in order for the liquid to be recovered in its bottom part. Cade oil is the most well known example of this method of production.

The cold expression production method is applied only to Citrus fruits (Citrus spp.) by mechanical processes at ambient temperature. The principle of the method is as follows: the peel is shredded and the contents of the secretory cavities which have been ruptured are recovered by a physical process. The conventional process consists in exerting an abrasive action over the entire surface of the fruit under a stream of water. After removal of the solid waste, the essential oil is separated from the aqueous phase by centrifugation. The majority of industrial plants in fact allow the simultaneous or sequential recovery of the fruit juices and of the essential oil.

Essential oils are generally volatile and liquid at ambient temperature, which differentiates them from “fixed” oils. They are more or less coloured and their density is generally lower than that of water. They have a high refractive index and the majority deviate polarized light.

They are soluble in fats and in standard organic solvents, can be steam distilled and exhibit a very low solubility in water.

Mention may be made, among the essential oils which can be used according to the invention, of those obtained from plants belonging to the following botanical families: Abietaceae or Pinaceae, for example conifers; Amaryllidaceae; Anacardiaceae; Annonaceae, for example ylang-ylang; Apiaceae, for example umbellifers, in particular dill, Angelica, coriander, sea fennel, carrot or parsley; Araceae; Aristolochiaceae; Asteraceae, for example Achillea, Artemisia, camomile, Helichrysum; Betulaceae; Brassicaceae; Burseraceae, for example frankincense; Caryophyllaceae; Canellaceae; Caesalpiniaceae, for example Copaifera (copaiba); Chenopodiaceae; Cistaceae, for example Cistus; Cyperaceae; Dipterocarpaceae; Ericaceae, for example Gaultheria (wintergreen); Euphorbiaceae; Fabaceae; Geraniaceae, for example Geranium; Guttiferae; Hamamelidaceae; Hernandiaceae; Hypericaceae, for example St. John's wort; Iridaceae; Juglandaceae; Lamiaceae, for example thyme, oregano, Monarda, savory, basil, marjorams, mints, patchouli, lavenders, sages, catnip, rosemary, hyssop or balm; Lauraceae, for example Ravensara, bay, rosewood, cinnamon, Litsea; Liliaceae, for example garlic; Magnoliaceae, for example Magnolia; Malvaceae; Meliaceae; Monimiaceae; Moraceae, for example hemp or hop; Myricaceae; Myristicaceae, for example nutmeg; Myrtaceae, for example Eucalyptus, tea tree, Melaleuca quinquenervia, cajuput, Backhousia, clove, myrtle; Oleaceae; Piperaceae, for example pepper; Pittosporaceae; Poaceae, for example Citronella grass, lemon grass, vetiver; Polygonaceae; Ranunculaceae; Rosaceae, for example roses; Rubiaceae; Rutaceae, for example the whole Citrus family; Salicaceae; Santalaceae, for example sandalwood; Saxifragaceae; Schisandraceae; Styracaceae, for example benzoin; Thymelaeaceae, for example agarwood; Tiliaceae; Valerianaceae, for example valerian, nard; Verbenaceae, for example lantana, Verbena; Violaceae; Zingiberaceae, for example galangal, turmeric, cardamom, ginger; Zygophyllaceae.

Mention may also be made of essential oils extracted from flowers (lily, lavender, rose, jasmine, ylang-ylang, neroli), from stalks and leaves (patchouli, Geranium, bitter orange), from fruits (coriander, anise, cumin, juniper berry), from fruit peels (bergamot, lemon, orange), from roots (Angelica, celery, cardamom, iris, sweet flag, ginger), from wood (pinewood, sandalwood, guaiac, pink cedar, camphor), from grasses and graminae (tarragon, rosemary, basil, lemon grass, sage, thyme), from needles and branches (spruce, fir, pine, dwarf pine) or from resins and balms (Galbanum, elemi, benzoin, myrrh, olibanum, Opopanax).

Examples of fragrancing substances are in particular: geraniol, geranyl acetate, farnesol, borneol, bornyl acetate, linalol, linalyl acetate, linalyl propionate, linalyl butyrate, tetrahydrolinalol, citronellol, citronellyl acetate, citronellyl formate, citronellyl propionate, dihydromyrcenol, dihydromyrcenyl acetate, tetrahydromyrcenol, terpineol, terpinyl acetate, nopol, nopyl acetate, nerol, neryl acetate, 2-phenylethanol, 2-phenylethyl acetate, benzyl alcohol, benzyl acetate, benzyl salicylate, styrallyl acetate, benzyl benzoate, amyl salicylate, dimethylbenzylcarbinol, trichloromethylphenylcarbinyl acetate, p-tert-butylcyclohexyl acetate, isononyl acetate, vetiveryl acetate, vetiverol, α-hexylcinnamaldehyde, 2-methyl-3-(p-tert-butylphenyl)propanal, 2-methyl-3-(p-isopropylphenyl)propanal, 3-(p-tert-butylphenyl)propanal, 2,4-dimethylcyclohex-3-enylcarboxaldehyde, tricyclodecenyl acetate, tricyclodecenyl propionate, 4-(4-hydroxy-4-methylpentyl)-3-cyclohexenecarboxaldehyde, 4-(4-methyl-3-pentenyl)-3-cyclohexenecarboxaldehyde, 4-acetoxy-3-pentyltetrahydropyran, 3-carboxymethyl-2-pentylcyclopentane, 2-(n-heptyl)cyclopentanone, 3-methyl-2-pentyl-2-cyclopentenone, menthone, carvone, tagetone, geranylacetone, n-decanal, n-dodecanal, 9-decen-1-ol, phenoxyethyl isobutyrate, phenylacetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, anisonitrile, anisaldehyde, heliotropin, coumarin, eugenol, vanillin, diphenyl ether, citral, citronellal, hydroxycitronellal, damascone, ionones, methylionones, isomethylionones, solanone, irones, cis-3-hexenol and its esters, indane musk compounds, tetralin musk compounds, isochroman musk compounds, macrocyclic ketones, macrolactone musk compounds, aliphatic musk compounds, ethylene brassylate and their mixtures.

In particular, a fragranced composition according to the invention can comprise one or more fragrancing substances which are natural or of natural origin.

A fragranced composition according to the invention can in particular have a content of fragrancing substance(s) which is(are) natural or of natural origin that is greater than the content of fragrancing substance(s) of synthetic origin.

According to a preferred embodiment of the invention, use is made of a mixture of different fragrancing substances which generate in common a note which is pleasant to the user.

Thus, according to a preferred embodiment, the fragranced composition comprises at least a mixture of fragrancing substances, in particular of at least two distinct fragrancing substances, with respect to the total weight of the composition, and preferably of at least three distinct fragrancing substances.

The fragrancing substances will preferably be chosen so that they produce notes (top, middle and base) in the following families: hesperidic, aromatic, floral notes, in particular pink flowers and white flowers, spicy, woody, gourmand, chypre, fougère, leather and musk.

For obvious reasons, the amount of fragrancing substance(s) present in a composition according to the invention is liable to vary significantly with regard to the odour or the odorous intensity sought by its presence.

By way of illustration, a fragranced composition according to the invention can comprise at least 0.5% by weight, in particular at least 1% by weight, more particularly from 1% to 35% by weight, preferably from 5% to 30% by weight, more preferentially from 10% to 25% by weight, of fragrancing substance(s), with respect to the total weight of said composition.

The fragrancing substances can be introduced into a fragranced composition in accordance with the invention in the form of a fragrance concentrate.

The fragrance concentrate can be a concrete or an absolute, preferably an absolute.

Thus, a fragranced composition according to the invention comprises particularly at least 0.5% by weight, in particular at least 1% by weight, more particularly from 1% to 35% by weight, preferably from 5% to 30% by weight, more preferentially from 10% to 25% by weight, of fragrance concentrate, with respect to the total weight of the composition.

### Additional UV Screening Agents

A fragranced composition according to the invention can also comprise one or more additional UV screening agent(s), distinct from the organic UV screening agents b) chosen from dibenzoylmethane derivatives which are defined above. The additional UV screening agent(s) can in particular be chosen from UV-A screening agents and/or UV-B screening agents.

The UV screening agents can be chosen from organic UV screening agents, which are hydrophilic or hydrophobic, and/or inorganic UV screening agents.

Preferentially, they are chosen from hydrophilic or hydrophobic organic UV screening agents.

The term “UV-B screening agent” is understood to denote any compound which screens out (or absorbs) ultraviolet (UV) radiation in the wavelength range extending from 280 nm to 320 nm.

The term “UV-A screening agent” is understood to denote any compound which screens out (or absorbs) ultraviolet (UV) radiation in the wavelength range extending from 320 nm to 400 nm. A distinction may be made between short UV-A screening agents (which absorb rays at a wavelength of between 320 and 340 nm) and long UV-A screening agents (which absorb rays at a wavelength between 340 and 400 nm).

The term “hydrophilic UV screening agent” means any organic or inorganic cosmetic or dermatological compound which screens out UV radiation liable to be completely dissolved in the molecular state in a liquid aqueous phase or else to be solubilized in colloidal form (for example in micellar form) in a liquid aqueous phase.

The term “hydrophobic UV screening agent” means any cosmetic or dermatological screening agent liable to be completely dissolved in the molecular state in a liquid fatty phase or else to be solubilized in colloidal form (for example in micellar form) in a liquid fatty phase.

Hydrophobic Organic UV-B and/or UV-A Screening Agents

The UV screening agents can be chosen from hydrophobic organic UV-B and/or UV-A screening agents chosen from:
i) para-aminobenzoic acid derivatives,
ii) cinnamic derivatives,
iii) salicylic derivatives,
iv) 3,3-diphenylacrylate and 4,4-diarylbutadiene derivatives,
v) benzophenone derivatives,
vi) benzylidenecamphor derivatives,
vii) phenylbenzotriazole derivatives,
viii) triazine derivatives,
ix) anthranilic derivatives,
x) imidazoline derivatives,
xi) benzalmalonate derivatives,
xii) merocyanine derivatives, and their mixtures.

More particularly, the hydrophobic organic UV-B and/or UV-A screening agents can be chosen from:
i) para-aminobenzoic acid (PABA) derivatives or esters, in particular of formula (IV), and also their optical and geometrical isomers and their solvates:

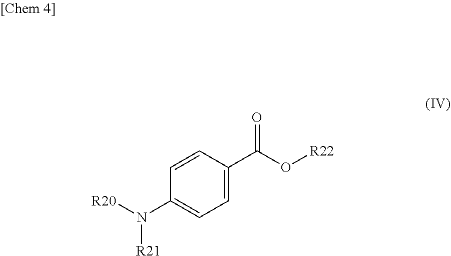

in which:
R20 and R21, which are identical or different, preferably identical, represent a hydrogen atom or a (C₁-C₆)alkyl, in particular (C₁-C₄)alkyl, group, such as methyl, ethyl, propyl, n-butyl and isobutyl;
R22 represents a group chosen from the groups of formulae (A) to (H) as defined above, or a (C₁-C₂₀)alkyl, in particular (C₁-C₁₀)alkyl, more particularly (C₁-C₆)alkyl, group, such as methyl, ethyl, propyl and butyl or a group of formula (A) as defined above.

In particular, the para-aminobenzoic acid (PABA) derivatives or esters can be chosen from the (C₁-C₆)alkyl) PABAs of formula (IV) in which:
R20 and R21, which are identical or different, preferably identical, represent a hydrogen atom or a (C₁-C₆)alkyl, in particular (C₁-C₄)alkyl, group, such as methyl, ethyl, propyl, n-butyl and isobutyl;
R22 represents a (C₁-C₂₀)alkyl, in particular (C₁-C₁₀)alkyl, more particularly (C₁-C₆)alkyl, group, such as methyl, ethyl, propyl and butyl.

In particular, the para-aminobenzoic acid (PABA) derivatives or esters are chosen from Ethyl PABA, Ethyl Dihydroxypropyl PABA, Ethylhexyl Dimethyl PABA, for example that sold under the name Escalol 507 by ISP, and their mixtures.

More particularly, the para-aminobenzoic acid (PABA) derivatives or esters can be chosen from Ethyl PABA, Ethyl Dihydroxypropyl PABA or their mixtures.
ii) cinnamic derivatives or esters, in particular of formula (V), and also their optical and geometrical isomers and their solvates:

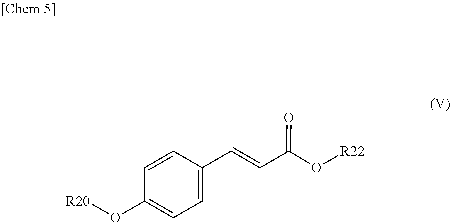

in which:
R20 represents a hydrogen atom or a (C₁-C₆)alkyl, in particular (C₁-C₄)alkyl, group, such as methyl; and
R22 represents a group chosen from the groups of formulae (A) to (H) as defined above, or a (C₁-C₂₀)alkyl, in particular (C₁-C₁₀)alkyl, more particularly (C₁-C₆)alkyl, group, such as methyl, ethyl, isopropyl, butyl and isoamyl.

In particular, the cinnamic derivatives or esters can be chosen from the compounds of formula (V) in which:
R20 represents a hydrogen atom or a (C₁-C₆)alkyl, in particular (C₁-C₄)alkyl, group, such as methyl;
R22 represents a (C₁-C₂₀)alkyl, in particular (C₁-C₁₀)alkyl, more particularly (C₁-C₆)alkyl, group, such as methyl, ethyl, isopropyl, butyl and isoamyl.

In particular, the cinnamic derivatives or esters can be chosen from Ethylhexyl Methoxycinnamate, for example that sold under the trade name Parsol MCX by Hoffmann-La Roche, Isopropyl Methoxycinnamate, Isoamyl Methoxycinnamate, for example that sold under the trade name Neo Heliopan E 1000 by Haarmann and Reimer, Cinoxate, Diisopropyl Methylcinnamate, Glyceryl Ethylhexanoate Dimethoxycinnamate and their mixtures.

More particularly, the cinnamic derivatives or esters can be chosen from Isopropyl Methoxycinnamate, Isoamyl Methoxycinnamate, Cinoxate, Diisopropyl Methylcinnamate and their mixtures.

It can in particular be Isoamyl Methoxycinnamate.
iii) salicylic derivatives or esters, in particular of formula (VI), and also their optical and geometrical isomers and their solvates:

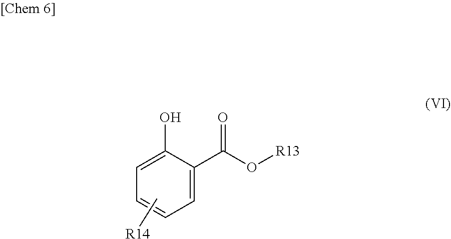

in which:
R13 represents a group chosen from the groups of formulae (A) to (H) as defined above, benzyl or branched C₄-C₂₀ alkyl, in particular C₆-C₁₂ alkyl, more preferentially branched C₈-C₁₀ alkyl; more particularly, R13 represents a group of formula (A) or benzyl;
R14 represents a hydrogen atom, a linear or branched C₁-C₂₀ alkyl radical, optionally forming rings, in particular represents a group chosen from the groups of formulae (A) to (H) as defined above, preferably a hydrogen atom.

In particular, the salicylic derivatives or esters can be chosen from the compounds of formula (VI) in which:
R13 represents a group chosen from the groups of formulae (A) to (H) as defined above or branched C₄-C₂₀ alkyl, in particular C₆-C₁₂ alkyl, more preferentially branched C₈-C₁₀ alkyl; more particularly, R13 represents a C₆ to C₁₆ alkyl radical substituted by at least one methyl, ethyl, propyl, isopropyl and/or tert-butyl radical;
R14 represents a hydrogen atom, a linear or branched C₁-C₂₀ alkyl radical, if appropriate cyclic, in particular represents a group chosen from the groups of formulae (A) to (H) and preferably is a hydrogen atom.

The salicylic derivatives or esters can in particular be chosen from Homosalate, for example that sold under the name Eusolex HMS by Rona/EM Industries, Ethylhexyl Salicylate, for example that sold under the name Neo Heliopan OS by Haarmann and Reimer, benzyl salicylate, Dipropylene Glycol Salicylate, in particular that sold under the name Dipsal by Scher, TEA Salicylate, in particular that sold under the name Neo Heliopan TS by Symrise, and their mixtures.

Preferably, the salicylic acid derivative is 2-ethylhexyl salicylate, in particular that corresponding to the commercial product Neo Heliopan OS.
iv) 3,3-diphenylacrylate derivatives or esters, in particular of formula (VII), and also their optical and geometrical isomers and their solvates, and 4,4-diarylbutadiene derivatives or diesters, in particular of formula (VII′), and also their optical and geometrical isomers and their solvates:

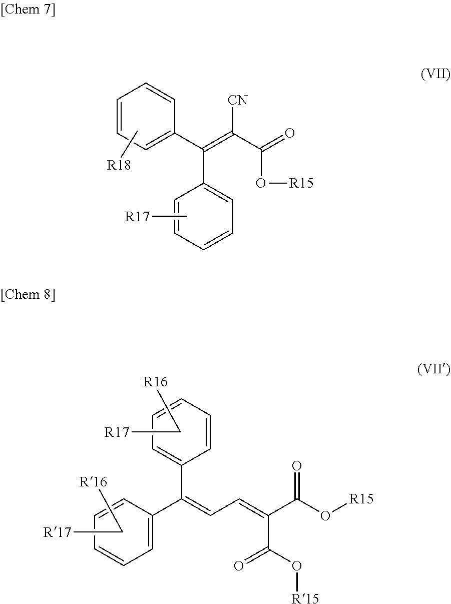

in which:
R15 and R′15, which are identical or different, represent a group chosen from the groups of formulae (A) to (H) as defined above, or a linear or branched C₁ to C₆ alkyl group, such as methyl, ethyl, propyl, isopropyl or tert-butyl, preferably ethyl, hexyl or a group of formula (A) as defined above;
R16, R′16, R17 and R′17, which are identical or different, represent a hydrogen atom, a linear or branched C₁-C₁₂ alkyl group or a linear or branched C₁-C₁₆ alkoxy radical; preferably, R16, R′16, R17 and R′17 represent a hydrogen atom or a C₁-C₄ alkyl group, such as methyl.

In particular, the 3,3-diphenylacrylate derivatives or esters can be chosen from the compounds of formula (VII) in which:
R15 represents a linear or branched, preferably linear, C₁ to C₆ alkyl group, such as methyl, ethyl, propyl or n-butyl, more preferentially ethyl; and
R16 and R17, which are identical or different, represent a hydrogen atom, a linear or branched C₁-C₁₀ alkyl group or a linear or branched C₁-C₁₀ alkoxy radical, preferably represent a hydrogen atom.

In particular, the 3,3-diphenylacrylate derivatives or esters can be chosen from 3,3-diphenyl-2-cyanoacrylate derivatives, in particular of formula (VII), and also their optical or geometrical isomers and their solvates, in which:
R15 represents a group chosen from the groups of formulae (A) to (H) as defined above, in particular a linear or branched C₆ to C₁₆ alkyl group, substituted by at least one methyl, ethyl, propyl, isopropyl or tert-butyl radical;
R16 and R17, which are identical or different, represent a hydrogen atom, a linear or branched C₁-C₁₂ alkyl group or a linear or branched C₁-C₁₆ alkoxy radical; preferentially, R16 and R17 represent a hydrogen atom.

Preferably, R15 represents a C₆ to C₈ alkyl radical substituted by at least one ethyl, propyl or isopropyl radical; more particularly, R15 represents a C₆ alkyl radical substituted by at least one ethyl, propyl or isopropyl radical; more preferentially still, R15 represents a group of formula (A).

In particular, it concerns Octocrylene, for example that sold under the trade name Uvinul N539 by BASF, Etocrylene, such as, for example, that sold under the trade name Uvinul N35 by BASF, and the following screening agents (1), (2) and (3) and their mixtures:

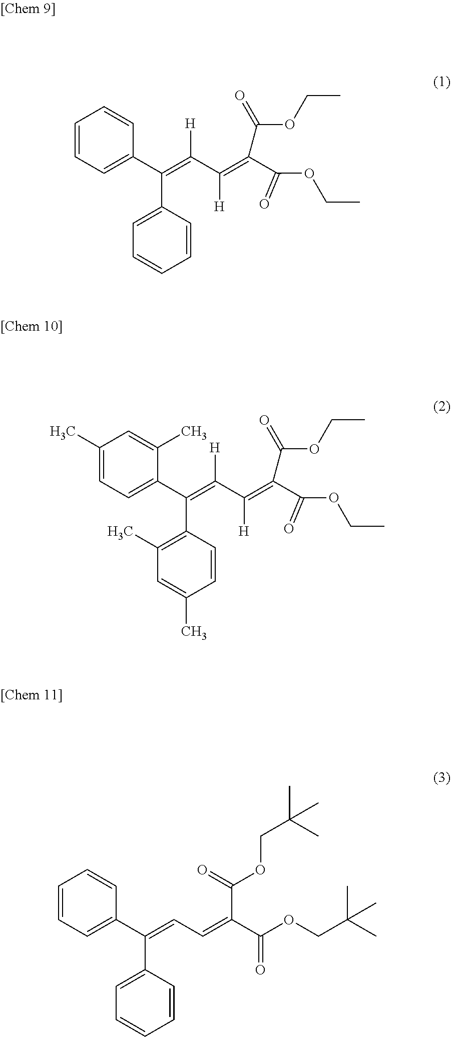

Etocrylene, such as, for example, that sold under the trade name Uvinul N35 by BASF, is very particularly suitable for the invention.
v) benzophenone derivatives or esters, in particular of formula (VIII), and also their optical and geometrical isomers and their solvates:

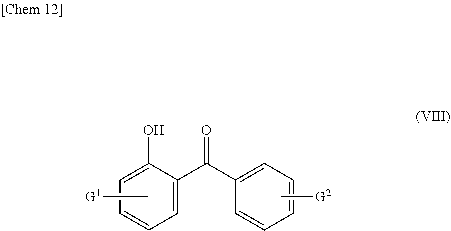

in which:
G¹ represents a hydrogen atom, an R1R2N— or R1-O— group, with R1 and R2, which are identical or different, representing a hydrogen atom, a linear or branched C₁-C₁₀ alkyl group, preferably a linear C₁-C₈ alkyl group, or else R1 and R2 form, together with the nitrogen atom which carries them, a saturated or unsaturated, preferably saturated, 5- or 6-membered heterocycle, such as pyrrolidino, piperazino, piperidino or morpholino;
G² represents a hydrogen atom or a hydroxyl group, a (C₁-C₄)alkyl group, such as methyl, or a —C(O)—O—R3 group with R3 representing a linear or branched C₁-C₁₂ carbon chain, optionally forming one or more rings, in particular a (C₄-C₁₀)alkyl group, preferably a (C₆-C₁₀)alkyl group, which is linear or branched, preferably linear, such as n-butyl, preferably a —C(O)—O—R3 group; in particular, G² is in the ortho position with respect to the carbonyl;
G¹ can be in the 3 or 5 position with respect to the carbonyl (in the meta position with respect to the hydroxyl or the para position with respect to the hydroxyl).

Preferably, G¹ represents an R1R2N— group and the R1 and R2 radicals are chosen from a hydrogen atom, a linear or branched C₁-C₄ alkyl group, such as methyl, ethyl, propyl, isopropyl, tert-butyl or pentyl optionally substituted in the 2 or 3 position by a methyl or ethyl group.

Preferably, R1 and R2 are chosen from an ethyl, propyl and butyl.

When R1 and R2 form, together with the nitrogen atom which carries them, a heterocycle, in particular a morpholino, pyrrolidino or piperidino group optionally substituted by one or more (C₁-C₄)alkyl groups, such as methyl. Preferably, R1 and R2 form, together with the nitrogen atom which carries them, a morpholino, pyrrolidino or piperidino group.

It very particularly concerns Benzophenone-3 or Oxybenzone, for example that sold under the trade name Uvinul M40 by BASF, Benzophenone-4, for example that sold under the trade name Uvinul MS40 by BASF, Benzophenone-8, for example that sold under the trade name Spectra-Sorb UV-24 by American Cyanamid, Benzophenone-12, n-hexyl 2-(4-diethylamino-2-hydroxybenzoyl)benzoate and their mixtures.

In particular, the benzophenone derivatives or esters can be chosen from the compounds of formula (VIII′):

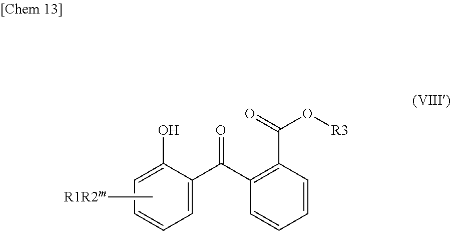

in which:
R1 and R2, which are identical or different, represent a hydrogen atom, a linear or branched C₁-C₈ alkyl group, preferably a C₁-C₆ alkyl group, or else R1 and R2 form, together with the nitrogen atom which carries them, a saturated or unsaturated, preferably saturated, 5- or 6-membered heterocycle, such as pyrrolidino, piperazino, piperidino or morpholino;
R3 represents a linear or branched C₁-C₁₂ carbon chain, optionally forming one or more rings, in particular a (C₄-C₁₀)alkyl group, preferably a (C₆-C₁₀)alkyl group, which is linear or branched, preferably linear, such as n-butyl; and
NR1R2 can be in the 3 or 5 position of the phenolic ring (in the meta position with respect to the hydroxyl).

Preferably, the R1 and R2 radicals are chosen from a hydrogen atom, a linear or branched C₁-C₄ alkyl group, such as methyl, ethyl, propyl, isopropyl, tert-butyl or pentyl optionally substituted in the 2 or 3 position by a methyl or ethyl group.

Preferably, R1 and R2 are chosen from an ethyl, propyl and butyl group.

When R1 and R2 form, together with the nitrogen atom which carries them, a heterocycle, in particular a morpholino, pyrrolidino or piperidino group optionally substituted by one or more (C₁-C₄)alkyl groups, such as methyl. Preferably, R1 and R2 form, together with the nitrogen atom which carries them, a morpholino, pyrrolidino or piperidino group.

The benzophenone derivatives can in particular be chosen from Benzophenone-1, in particular sold under the trade name Uvinul 400 by BASF; Benzophenone-2, in particular sold under the trade name Uvinul D50 by BASF; Benzophenone-3 or Oxybenzone, in particular sold under the trade name Uvinul M40 by BASF; Benzophenone-6, in particular sold under the trade name Helisorb 11 by Norquay; Benzophenone-8, in particular sold under the trade name Spectra-Sorb UV-24 by American Cyanamid; Benzophenone-10; Benzophenone-11; Benzophenone-12.

The benzophenone derivatives are preferably chosen from n-hexyl 2-(4-diethylamino-2-hydroxybenzoyl)benzoate, for example sold under the trade name Uvinul A+by BASF.
vi) benzylidenecamphor derivatives, in particular of formula (IX), and also their optical and geometrical isomers and their solvates:

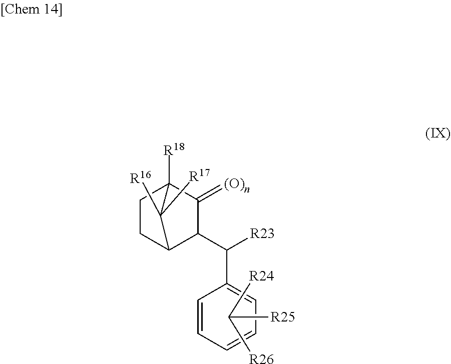

in which:
R¹⁶, R¹⁷, R⁸ and n are as defined in the preceding formula (E);
R23 represents a hydrogen atom or a (C₁-C₄)alkyl group, preferably a hydrogen atom;
R24 represents a hydrogen atom or a (C₁-C₆)alkyl, (C₁-C₆)alkoxy, (di)(C₁-C₆)(alkyl)amino or R27-C(O)—X—(CH₂)ₚ— group with R27 representing a (C₁-C₄)alkyl or (C₂-C₄)alkenyl group, X representing an oxygen atom or NH, p being an integer of between 0 and 4, in particular 1; in particular, R24 represents (C₁-C₆)alkyl, such as methyl; preferably, R24 is in the para position with respect to the styryl group; and
R25 and R26, which are identical or different, represent a hydrogen atom or a hydroxyl, (C₁-C₆)alkyl, (C₁-C₆)alkoxy or (di)(C₁-C₆)(alkyl)amino group, preferably a hydrogen atom; preferably, R25 et R26 are in the meta position with respect to the styryl group;

In particular, this screening agent is chosen from 3-Benzylidene Camphor, for example that sold under the name Mexoryl SD by Chimex, 4-Methylbenzylidene Camphor, for example that sold under the name Eusolex 6300 by Merck, Polyacrylamidomethyl Benzylidene Camphor, for example that manufactured under the name Mexoryl SW by Chimex, and their mixtures.
vii) phenyl benzotriazole derivatives, in particular chosen from those of formula (X), and also their optical and geometrical isomers and their solvates:

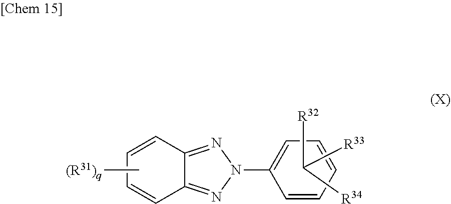

in which:
q has the value 0, 1, 2, 3 or 4; preferably, q is zero;
R³¹, when q has the value 1, 2, 3 or 4, represents a linear or branched (C₁-C₁₀)alkyl or linear or branched (C₁-C₁₀)alkoxy group;
R³² represents a hydrogen atom or a hydroxyl, (di)(C₁-C₆)(alkyl)amino, linear or branched (C₁-C₁₀)alkyl or linear or branched (C₁-C₁₀)alkoxy group, preferably a hydroxyl group; more particularly, R³² is located, on the phenyl group, in the ortho position with respect to the benzotriazolyl group;
R³³ represents a hydrogen atom, a group chosen from the groups of formulae (A) to (H) as defined above, a group chosen from: i) -(ALK)ₜ-(A), ii) -(ALK)ₜ—(B), iii) -(ALK)ₜ—(C), iv) -(ALK)ₜ-(D), v) -(ALK)ₜ-(E), vi) -(ALK)ₜ—(F) or -(ALK)ₜ—(F′), vii) -(ALK)ₜ-(G), and viii) -(ALK)ₜ—(H), with t having the value 0 or 1, ALK representing a linear or branched, preferably branched, (C₁-C₆)alkylene group, more preferentially a linear or branched, preferably branched, (C₁-C₄)alkylene group, and (A) to (H) representing a group chosen from the groups of formulae (A) to (H) as defined above, a following group (J):

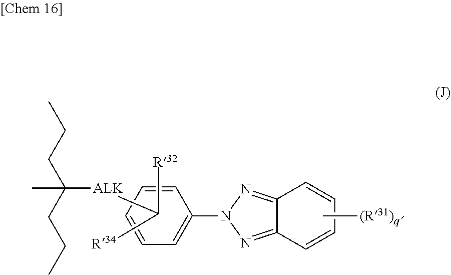

in which ALK is as defined above, preferably a methylene group, and R′³¹, R′³², R′³⁴ and q′ are as defined respectively for R³¹, R³², R³⁴ and q; preferentially, R′³¹=R³¹, R′³²=R³², R′³⁴=R³⁴ and q′=q;
preferably, R³³ is located, on the phenyl group, in the meta position with respect to the benzotriazolyl group;
R³⁴ represents a hydrogen atom or a linear or branched, preferably branched, (C₁-C₁₂)alkyl group, more preferentially chosen from the groups (A) and (B) as defined above, more preferentially still (B), such as ^{t}-Bu-CH₂—C(CH₃)₂—.

The phenyl benzotriazole derivatives can be chosen from 2-(2H-benzotriazol-2-yl)phenol derivatives, in particular chosen from those of formula (XI), and also their optical isomers and their solvates:

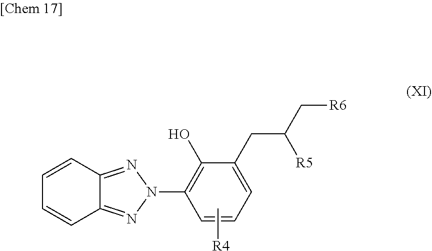

in which:
R4 represents a saturated or unsaturated, preferably saturated, linear or branched C₁-C₁₂ carbon chain optionally forming one or more saturated or unsaturated rings; preferably, R4 represents a linear or branched (C₁-C₆)alkyl, in particular (C₁-C₄)alkyl, group, such as methyl; more preferentially, the R4 radical is located, on the phenyl, in the para position with respect to the hydroxyl group;
R5 represents a saturated or unsaturated, preferably saturated, linear or branched C₁-C₆ carbon chain; R5 more particularly represents a linear or branched (C₁-C₆)alkyl, in particular (C₁-C₄)alkyl, group and preferably methyl;
R6 represents a group chosen from the groups of formulae (A) to (H) as defined above; preferably, R6 represents RR′R″Si— with R, R″ and R″′, which are identical or different, representing a (C₁-C₆)alkyl, (C₁-C₆)alkoxy or tri(C₁-C₄)alkylsiloxy group; preferably, R6 represents a group chosen from the groups of formula (C) or (D); more preferentially, R6 is of formula (XII):

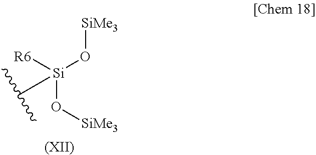

with R6 representing a linear or branched C₁-C₆ alkyl, in particular such as methyl.

Preferably, R4, R5 and R6 represent a methyl, ethyl, propyl or isopropyl radical and more particularly a methyl.

In particular, the phenyl benzotriazole derivatives can be chosen from silatrizole, for example Mexoryl XL, Drometrizole Trisiloxane, for example that sold under the name Silatrizole by Rhodia Chimie, Methylene Bis-Benzotriazolyl Tetramethylbutylphenol, for example that sold, in the solid form, under the trade name Mixxim BB/100 by Fairmount Chemical or, in the micronized form, in aqueous dispersion, under the trade name Tinosorb M by Ciba Specialty Chemicals, 2-(2H-benzotriazol-2-yl)-4,6-bis(1-methyl-1-phenylethyl)phenol, for example that sold under the name HMPB2 or Eversorb 89.

It can preferably concern silatrizole.
viii) triazine derivatives, in particular chosen from those of formula (XIII) or (XIII′), and also their optical or tautomeric isomers and their solvates:

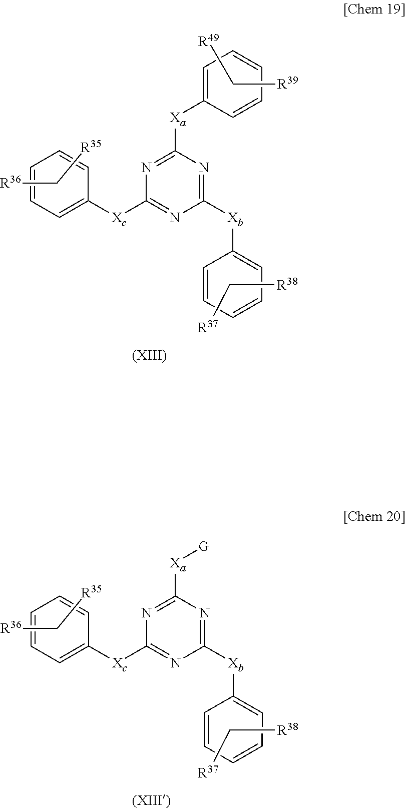

in which:
Xₐ, X_{b} and X_{c}, which are identical or different, preferably identical, represent a bond, an oxygen atom or —N(Rₐ)— with Rₐ representing a hydrogen atom or a (C₁-C₄)alkyl group; preferably, Xₐ, X_{b} and X_{c} are identical and represent a bond or —N(H)—, more preferentially —N(H)—;
R³⁵, R³⁷ and R³⁹, which are identical or different, represent a hydrogen atom or a hydroxyl, linear or branched (C₁-C₆)alkyl, linear or branched (C₁-C₆)alkoxy or (di)(C₁-C₆)(alkyl)amino group, preferably a hydrogen atom or a hydroxyl group;
   in particular, R³⁵, R³⁷ and R³⁹ are located in the ortho positions of the phenyls bonded to the triazine group;
G represents a group chosen from the groups of formulae (A) to (H) as defined above, preferably a group of formula (A) as defined above;
R³⁶, R³⁸ and R⁴⁰, which are identical or different, represent a hydrogen atom or a group chosen from a) linear or branched (C₁-C₆)alkyl, b) (C₁-C₆)alkylcarbonyl, c) linear or branched (C₁-C₆)alkoxy, such as methoxy, d) (C₁-C₆)alkoxycarbonyl, e) (di)(C₁-C₆)(alkyl)amino, f) (di)(C₁-C₄)(alkyl)aminocarbonyl, in particular such as ^{t}-butylaminocarbonyl, g) (C₁-C₄)(alkyl)carbonylamino, h) —C(R⁴¹)=C[C(O)—O—R⁴²]₂, i) —(X′)ₜ-(A), j) —(X′)ₜ—(B), k) —(X′)ₜ—(C), l) —(X′)ₜ-(D), m) —(X′)ₜ-(E), n) —(X′)ₜ—(F) or —(X′)ₜ—(F′), o) —(X′)ₜ-(G), p) —(X′)ₜ—(H), q) 2-(benz)oxazolyl optionally substituted by one or more linear or branched, preferably branched, (C₁-C₆)alkyl groups, r) 2-(benz)imidazolyl optionally substituted by one or more linear or branched, preferably branched, (C₁-C₆)alkyl groups, s) 2-(benzo)triazolyl optionally substituted by one or more linear or branched, preferably branched, (C₁-C₆)alkyl groups; with (A) to (H) being a group of formulae (A) to (H) as defined above; R⁴¹ representing a hydrogen atom or a (C₁-C₄)alkyl group, R⁴² representing a linear or branched, preferably branched, (C₁-C₆)alkyl group, such as t-butyl, t having the value 0 or 1, preferably 1, X′ representing an ALK group as defined above or Y; with Y representing an oxygen atom, —N(H)—, —C(O)—, —C(O)—O—, —O—C(O)—, —C(O)—N(H)—, —N(H)—C(O)—, —O—C(O)—N(H)— or —N(H)—C(O)—O—, or —N(H)—C(O)—N(H)—; preferably, Y represents an ester;
more particularly, the R³⁶ groups represent a linear or branched (C₁-C₆)alkoxy group, such as methoxy, or —C(O)—O-(A), R³⁸ and R⁴⁰, which are identical or different, preferably identical, represent —O-(A) or —C(O)—O-(A) with (A) being a group of formula (A) as defined above;
in particular, R³⁶, R³⁸ and R⁴⁰ are located in the para positions of the phenyls bonded to the triazine group;
it being understood that at least two R³⁵ to R⁴⁰ groups are other than a hydrogen atom.

In particular, the triazine derivatives can be chosen from 2,4-bis(hydroxyphen-2-yl)-1,3,5-triazine derivatives, preferably those of formula (XIV), and also their optical isomers and their solvates:

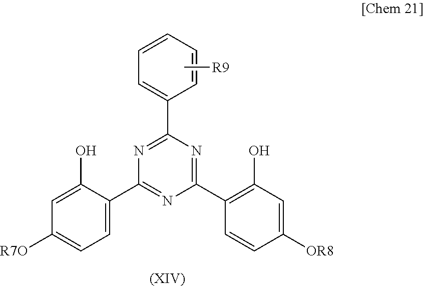

in which:
R₇ and R₈, which are identical or different, preferably identical, represent a group chosen from the groups of formulae (A) to (H) as defined above or a branched C₄-C₂₀, in particular C₆-C₁₂, more preferentially C₈-C₁₀, alkyl optionally forming a ring or several rings; preferably, R₇ and R₈ represent a group chosen from the groups of formula (A) as defined above; and
R₉ represents a linear or branched C₁-C₆ alkoxy, such as methoxy.

In particular, the triazine derivatives can also be chosen from 2,4,6-tri(anilino)-1,3,5-triazine compounds, in particular of formula (XV), and also their optical isomers and their solvates:

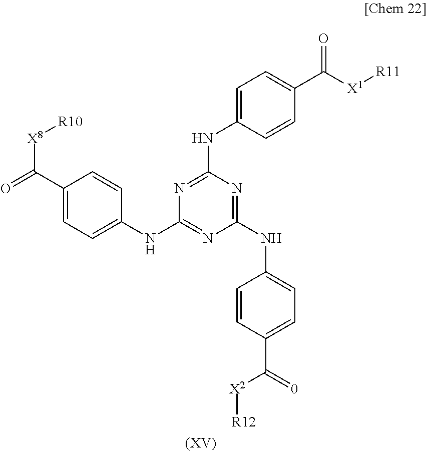

in which:
X¹, X² and X³, which are identical or different, preferably identical, represent an oxygen atom or an NR′12 group; preferably, X¹, X² and X³ represent an oxygen atom;
R₁₀, R₁₁ and R₁₂, which are identical or different, preferably identical, represent a group chosen from the groups of formulae (A) to (H) as defined above or branched C₄-C₂₀, in particular C₆-C₁₂, more preferentially C₈-C₁₀, alkyl; and
R′12 represents a hydrogen atom, a linear or branched C₇-C₂₀ alkyl radical, or a group chosen from the groups of formulae (A) to (H) as defined above, or branched C₄-C₂₀, in particular C₆-C₂, more preferentially C₈-C₁₀, alkyl; in particular, R′12 represents a hydrogen atom or a group of formula (A);
with preferentially R₁₀, R₁₁ and R₁₂ representing a group of formula (A); in particular, the triazine derivatives of formula (XV) are chosen from tris(2-propylhexyl) 4,4′,4″-(1,3,5-triazine-2,4,6-triyltriimino)tribenzoate, tris(2-ethylhexyl) 4,4′,4″-(1,3,5-triazine-2,4,6-triyltriimino)tribenzoate (also denoted Ethylhexyl Triazone, Octyl Triazone or sold under the name Uvinul T150), tris(2-ethyl-2-propylhexyl) 4,4′,4″-(1,3,5-triazine-2,4,6-triyltriimino)tribenzoate, and tris(2-ethyloctyl) 4,4′,4″-(1,3,5-triazine-2,4,6-triyltriimino)tribenzoate and their mixtures.

In particular, the triazine derivatives can be chosen from Bis-Ethylhexyloxyphenol Methoxyphenyl Triazine, for example that sold under the trade name Tinosorb S by Ciba Geigy, Ethylhexyl Triazone, for example that sold under the trade name Uvinul T150 by BASF, Diethylhexyl Butamido Triazone, for example that sold under the trade name Uvasorb HEB by Sigma 3V, 2,4,6-tris(diisobutyl 4′-aminobenzalmalonate)-s-triazine, 2,4,6-tris(dineopentyl 4′-aminobenzalmalonate)-s-triazine, 2,4-bis(dineopentyl 4′-aminobenzalmalonate)-6-(n-butyl 4′-aminobenzoate)-s-triazine; 2,4-bis(n-butyl 4′-aminobenzoate)-6-(aminopropyltrisiloxane)-s-triazine; 2,4-bis[5-(1,1-dimethylpropyl)benzoxazol-2-yl(4-phenyl)imino]-6-(2-ethylhexyl)imino-1,3,5-triazine, for example that sold under the name Uvasorb K2A by Sigma 3V, tris(2-propylhexyl) 4,4′,4″-(1,3,5-triazine-2,4,6-triyltriimino)tribenzoate, tris(2-ethylhexyl) 4,4′,4″-(1,3,5-triazine-2,4,6-triyltriimino)tribenzoate, tris(2-ethyl-2-propylhexyl) 4,4′,4″-(1,3,5-triazine-2,4,6-triyltriimino)tribenzoate, tris(2-ethyloctyl) 4,4′,4″-(1,3,5-triazine-2,4,6-triyltriimino)tribenzoate and their mixtures.

Preferably, the triazine derivatives can be chosen from 2,2′-[6-(4-methoxyphenyl)-1,3,5-triazine-2,4-diyl]bis{5-[(2-ethylhexyl)oxy]phenol}, also denoted Bis-Ethylhexyloxyphenol Methoxyphenyl Triazine, Bemotrizinol or Tinosorb S, Anisotriazine and their mixtures. The screening agent sold under the name Tinosorb S is very particularly suitable.

Preferably, the triazine derivatives can be chosen from tris(2-ethylhexyl) 4,4′,4″-(1,3,5-triazine-2,4,6-triyltriimino)tribenzoate (Uvinul T150) and/or 2-ethylhexyl 4-[[4,6-bis[[4-(2-ethylhexoxy-oxomethyl)phenyl]amino]-1,3,5-triazin-2-yl]amino]benzoate.

Preferably, it concerns 2,4,6-tri(anilino)-1,3,5-triazine, in particular sold under the name Ethylhexyl Triazone or Uvinul T150.
ix) anthranilic derivatives, in particular of formula (XVI), and also their optical and geometrical isomers and their solvates:

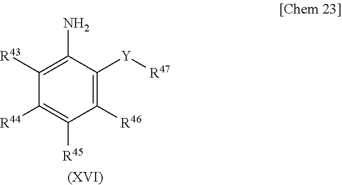

in which:
Y is as defined above, in particular —C(O)—O— or —O—C(O)—; preferably, Y represents —C(O)—O—;
R⁴³ to R⁴⁶, which are identical or different, preferably identical, represent a hydrogen atom or a (C₁-C₆)alkyl, (C₁-C₆)alkoxy or (di)(C₁-C₆)(alkyl)amino group, preferably a hydrogen atom;
R⁴⁷ represents a group of formulae (A) to (H) as defined above, preferably of formula (F) or (F′), more preferentially (F) such as menthyl;
   and in particular menthyl anthranilate, sold in particular under the trade name Neo Heliopan MA by Symrise.
x) imidazoline derivatives, in particular those of formula (XVII), and also their optical and geometrical isomers, their tautomers and their solvates:

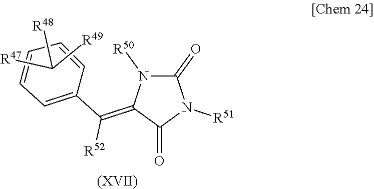

in which:
R⁴⁷, R⁴⁸ and R⁴⁹, which are identical or different, represent a hydrogen atom or a linear or branched (C₁-C₆)alkyl or linear or branched (C₁-C₆)alkoxy group; in particular, R⁴⁷ represents a hydrogen atom and R⁴⁸ and R⁴⁹ represent a (C₁-C₄)alkoxy, in particular methoxy, group;
R⁵⁰ represents a hydrogen atom or a linear or branched (C₁-C₆)alkyl group, preferably hydrogen;
R⁵¹ represents a hydrogen atom or a linear or branched (C₁-C₆)alkyl group, preferably hydrogen, -ALK′-(Y)ₜ-(A), -ALK′-(Y)ₜ—(B), -ALK′-(Y)ₜ—(C), -ALK′-(Y)ₜ-(D), -ALK′-(Y)ₜ-(E), -ALK′-(Y)ₜ—(F) or -ALK′-(Y)ₜ—(F′), -ALK′-(Y)ₜ-(G) and -ALK′-(Y)ₜ—(H) with t having the value 0 or 1, preferably 1, (A) to (H) representing a group of formulae (A) to (H) as defined above with Y as defined above; preferably, Y represents an ester, and more particularly —C(O)—O-(A);
   and in particular Ethylhexyl Dimethoxybenzylidene Dioxoimidazoline Propionate.
xi) benzalmalonate derivatives, in particular polyorganosiloxanes having a benzalmalonate function, especially Polysilicone-15, in particular sold under the trade name Parsol SLX by DSM Nutritional Products Inc., and dineopentyl 4′-méthoxybenzalmalonate.
xii) merocyanine derivatives, in particular octyl 5-N,N-diethylamino-2-phenylsulfonyl-2,4-pentadienoate.
   Hydrophilic Organic UV-B and/or UV-A Screening Agents

Mention may in particular be made, among hydrophilic organic UV-B and/or UV-A screening agents, of hydrophilic UV-A screening agents, such as the bis-benzazolyl derivatives as described in Patents EP 669 323 and U.S. Pat. No. 2,463,264, and more particularly the compound Disodium Phenyl Dibenzimidazotetrasulfonate, sold in particular under the trade name Neo Heliopan AP by Symrise.

Mention may also be made of Terephthalylidene Dicamphor Sulfonic Acid (sold, for example, under the trade name Mexoryl SX).

Mention may in particular be made, among hydrophilic organic UV-B and/or UV-A screening agents, of hydrophilic UV-B screening agents, such as:
the following p-aminobenzoic acid (PABA) derivatives: PABA; Glyceryl PABA; and PEG-25 PABA, in particular sold under the trade name Uvinul P25 by BASF;
Phenylbenzimidazole Sulfonic Acid, in particular sold under the trade name Eusolex 232 by Merck;
ferulic acid;
salicylic acid;
DEA methoxycinnamate;
Benzylidene Camphor Sulfonic Acid, in particular sold under the name Mexoryl SL by Chimex;
Camphor Benzalkonium Methosulfate, in particular sold under the name Mexoryl SO by Chimex.

### Inorganic UV Screening Agents

The composition according to the invention can also employ inorganic UV screening agents, which are generally uncoloured pigments. The pigments may or may not be coated.

Within the meaning of the present invention, the term “pigments” is understood to denote white or coloured and inorganic or organic solid particles.

The term “uncoloured” means that, to the naked eye, the pigment does not absorb in the visible spectrum and appears white or colourless

The coated pigments are pigments which have undergone one or more surface treatments of chemical, electronic, mechanochemical and/or mechanical nature with compounds as described, for example, in Cosmetics & Toiletries, February 1990, Vol. 105, pages 53-64, such as amino acids, beeswax, fatty acids, fatty alcohols, anionic surfactants, lecithins, sodium, potassium, zinc, iron or aluminium salts of fatty acids, metal alkoxides (titanium or aluminium alkoxides), polyethylene, silicones, proteins (collagen, elastin), alkanolamines, silicon oxides, metal oxides or sodium hexametaphosphate.

As is known, silicones are organosilicon polymers or oligomers having a linear or cyclic, branched or crosslinked, structure, of variable molecular weight, which are obtained by polymerization and/or polycondensation of suitably functionalized silanes, and consist essentially of a repetition of main units in which the silicon atoms are connected together by oxygen atoms (siloxane bond), optionally substituted hydrocarbon radicals being directly bonded via a carbon atom to said silicon atoms.

The term “silicones” also encompasses the silanes required for their preparation, in particular alkylsilanes.

The silicones used for the coating of the pigments suitable for the present invention are preferably chosen from the group containing alkylsilanes, polydialkylsiloxanes and polyalkylhydrosiloxanes. More preferentially still, the silicones are chosen from the group containing octyltrimethylsilane, polydimethylsiloxanes and polymethylhydrosiloxanes.

Of course, before being treated with silicones, the metal oxide pigments may have been treated with other surface agents, in particular with cerium oxide, alumina, silica, aluminium compounds, silicon compounds or their mixtures.

The coated pigments are, for example, titanium oxides coated:
with silica, in particular the product Sunveil from Ikeda and the product Eusolex T-Avo from Merck,
with silica and iron oxide, in particular the product Sunveil F from Ikeda,
with silica and alumina, in particular the products Microtitanium Dioxide MT 500 SA and Microtitanium Dioxide MT 100 SA from Tayca, Tioveil from Tioxide and Mirasun TiW 60 from Rhodia,
with alumina, in particular the products Tipaque TTO-55 (B) and Tipaque TTO-55 (A) from Ishihara and UVT 14/4 from Kemira,
with alumina and aluminium stearate, in particular the products Microtitanium Dioxide MT 100 TV, MT 100 TX, MT 100 Z and MT-01 from Tayca and the products Solaveil CT-10 W, Solaveil CT 100 and Solaveil CT 200 from Uniqema,
with silica, alumina and alginic acid, in particular the product MT-100 AQ from Tayca,
with alumina and aluminium laurate, in particular the product Microtitanium Dioxide MT 100 S from Tayca,
with iron oxide and iron stearate, in particular the product Microtitanium Dioxide MT 100 F from Tayca,
with zinc oxide and zinc stearate, in particular the product BR351 from Tayca,
with silica and alumina and treated with a silicone, in particular the products Microtitanium Dioxide MT 600 SAS, Microtitanium Dioxide MT 500 SAS and Microtitanium Dioxide MT 100 SAS from Tayca,
with silica, alumina and aluminium stearate and treated with a silicone, in particular the product STT-30-DS from Titan Kogyo,
with silica and treated with a silicone, in particular the product UV-Titan X 195 from Kemira or the product SMT-100 WRS from Tayca,
with alumina and treated with a silicone, in particular the products Tipaque TTO-55 (S) from Ishihara and UV Titan M 262 from Kemira,
with triethanolamine, in particular the product STT-65-S from Titan Kogyo,
with stearic acid, in particular the product Tipaque TTO-55 (C) from Ishihara,
with sodium hexametaphosphate, in particular the product Microtitanium Dioxide MT 150 W from Tayca.

Other titanium oxide pigments treated with a silicone are, for example, TiO₂ treated with octyltrimethylsilane, such as the product sold under the trade name T 805 by Degussa Silices, TiO₂ treated with a polydimethylsiloxane, such as the product sold under the trade name 70250 Cardre UF TiO₂SI₃ by Cardre, anatase/rutile TiO₂ treated with a polydimethylhydrosiloxane, such as the product sold under the trade name Microtitanium Dioxide USP Grade Hydrophobic by Color Techniques.

The uncoated titanium oxide pigments are sold, for example, by Tayca under the trade names Microtitanium Dioxide MT 500 B and Microtitanium Dioxide MT 600 B, by Degussa under the name P 25, by Wackher under the name Transparent Titanium Oxide PW, by Miyoshi Kasei under the name UFTR, by Tomen under the name ITS and by Tioxide under the name Tioveil AQ.

The uncoated zinc oxide pigments are, for example:
those sold under the name Z-Cote by Sunsmart;
those sold under the name Nanox by Elementis,
those sold under the name Nanogard WCD 2025 by Nanophase Technologies.

The coated zinc oxide pigments are, for example:
those sold under the name Z-Cote HP1, by Sunsmart (dimethicone-coated ZnO),
those sold under the name Zinc Oxide CS-5 by Toshibi (ZnO coated with polymethylhydrosiloxane),
those sold under the name Nanogard Zinc Oxide FN by Nanophase Technologies (as a 40% dispersion in Finsolv TN, C₁₂-C₁₅ alkyl benzoate),
those sold under the names Daitopersion Zn-30 and Daitopersion Zn-50 by Daito (dispersions in oxyethylenated polydimethylsiloxane/cyclopolymethylsiloxane containing 30% or 50% of zinc oxides coated with silica and polymethylhydrosiloxane),
those sold under the name NFD Ultrafine ZnO by Daikin (ZnO coated with perfluoroalkyl phosphate and copolymer based on perfluoroalkylethyl as a dispersion in cyclopentasiloxane),
those sold under the name SPD-Z1 by Shin-Etsu (ZnO coated with silicone-grafted acrylic polymer, dispersed in cyclodimethylsiloxane),
those sold under the name Escalol Z100 by ISP (alumina-treated ZnO dispersed in the ethylhexyl methoxycinnamate/PVP-hexadecene copolymer/methicone mixture),
those sold under the name Fuji ZnO-SMS-10 by Fuji Pigment (ZnO coated with silica and polymethylsilsesquioxane),
those sold under the name Nanox Gel TN by Elementis (ZnO dispersed at 55% in C₁₂-C₁₅ alkyl benzoate with hydroxystearic acid polycondensate).

The uncoated cerium oxide pigments are sold, for example, under the name Colloidal Cerium Oxide.

The uncoated iron oxide pigments are, for example, sold by Arnaud under the names Nanogard WCD 2002 (FE 45B), Nanogard Iron FE 45 BL AQ, Nanogard FE 45R AQ and Nanogard WCD 2006 (FE 45R) or by Mitsubishi under the name TY-220.

The coated iron oxide pigments are, for example, sold by Arnaud under the names Nanogard WCD 2008 (FE 45B FN), Nanogard WCD 2009 (FE 45B 556), Nanogard FE 45 BL 345 and Nanogard FE 45 BL or by BASF under the name Transparent Iron Oxide.

Mention may also be made of mixtures of metal oxides, in particular of titanium dioxide and of cerium dioxide, including the equal-weight mixture of titanium dioxide and cerium dioxide which are coated with silica, sold by Ikeda under the name Sunveil A, and also the mixture of titanium dioxide and zinc dioxide coated with alumina, silica and silicone, such as the product M 261 sold by Kemira, or coated with alumina, silica and glycerol, such as the product M 211 sold by Kemira.

The pigments can be introduced as is or in the form of a pigment paste, that is to say as a mixture with a dispersant, as described, for example, in the document GB-A-2 206 339.

According to a specific embodiment, the composition according to the invention does not employ inorganic UV screening agents.

According to a specific embodiment, the amount of the inorganic UV screening agent(s) can range from 0.01% to 20% by weight, with respect to the total weight of the composition. It ranges, for example, from 1% to 15% by weight, with respect to the total weight of the composition.

According to a preferred embodiment, the composition according to the invention furthermore employs one or more additional organic UV screening agents, preferably hydrophobic screening agents.

According to a preferred embodiment, the composition according to the invention employs one or more additional UV screening agent(s) chosen from hydrophobic organic UV-A screening agents, hydrophobic organic UV-B screening agents and/or hydrophobic organic mixed UV-A and UV-B screening agents.

More preferentially, the composition according to the invention furthermore employs one or more additional UV screening agents chosen from para-aminobenzoic acid derivatives, 3,3-diphenylacrylate and 4,4-diarylbutadiene derivatives, benzophenone derivatives, benzylidene camphor derivatives, triazine derivatives, anthranilic derivatives, imidazoline derivatives, benzalmalonate derivatives and their mixtures.

According to one embodiment, the amount of the additional organic UV screening agent(s) present in a composition according to the invention can range from 0.05% to 5% by weight, in particular from 0.1% to 0.5% by weight, with respect to the total weight of the composition.

In particular, the composition according to the invention comprises less than 1% by weight, preferably less than 0.5% by weight, more preferentially less than 0.1% by weight, in particular less than 0.01% by weight, of additional UV screening agents, indeed even is devoid of additional UV screening agents.

### Additional Anti-Oxidizing Agents

A fragranced composition according to the invention can additionally comprise one or more anti-oxidizing agent(s) distinct from the compounds of formula (I) or (II).

In particular, a fragranced composition according to the invention can also comprise one or more additional anti-oxidizing agent(s) distinct from the sulfur-comprising antioxidant compounds a) defined above and the vitamins defined below.

The anti-oxidizing agent can be of primary type or of secondary type, and can be chosen from hindered phenols, aromatic secondary amines, organophosphorus compounds, sulfur-comprising compounds, lactones, bisphenol acrylates and their mixtures.

Mention may in particular be made, among the antioxidants very particularly suitable for the invention, of butylated hydroxyanisole (BHA), tert-butylhydroquinone (TBHQ), 1,3,5-trimethyl-2,4,6-tris(3,5-di(tert-butyl)-4-hydroxybenzyl)benzene,octadecyl 3,5-di(tert-butyl)-4-hydroxycinnamate, pentaerythrityl tetra(di(t-butyl)hydroxycinnamate), such as that sold by BASF under the name Tinogard® TT, tetrakis-methylene-3-(3,5-di(tert-butyl)-4-hydroxyphenyl)propionate methane, octadecyl 3-(3,5-di(tert-butyl)-4-hydroxyphenyl)propionate, 2,5-di(tert-butyl)hydroquinone, 2,2-methylenebis(4-methyl-6-(tert-butyl)phenol), le 2,2-methylenebis(4-ethyl-6-(tert-butyl)phenol), 4,4-butylidenebis(6-(tert-butyl)-m-cresol), N,N-hexamethylenebis(3,5-di(tert-butyl)-4-hydroxyhydrocinnamamide), pentaerythrityl tetrakis(3-(3,5-di(tert-butyl)-4-hydroxyphenyl)propionate), in particular that sold by CIBA under the name Irganox® 1010, octadecyl 3-(3,5-di(tert-butyl)-4-hydroxphenyl)propionate, in particular that sold by CIBA under the name Irganox® 1076, 1,3,5-tris(3,5-di(tert-butyl)-4-hydroxybenzyl)-1,3,5-triazine-2,4,6(1H,3H,5H)trione, in particular that sold by Mayzo of Norcross, Ga., under the name BNX® 3114, di(stearyl) pentaerythritol diphosphite, tris(2,4-di(tert-butyl)phenyl) phosphite, in particular that sold by CIBA under the name Irgafos® 168, bis(2,4-di(tert-butyl)pentaerythritol diphosphite, in particular that sold by CIBA under the name Irgafos© 126, bis(2,4-bis[2-phenylpropan-2-yl]phenyl) pentaerythritol diphosphite, triphenyl phosphite, (2,4-di(tert-butyl)phenyl) pentaerythritol diphosphite, in particular that sold by GE Specialty Chemicals under the name UL Tranox® 626, tris(nonylphenyl) phosphite, in particular that sold by CIBA under the name Irgafos® TNPP, the 1:1 mixture of N,N-hexamethylenebis(3,5-di(tert-butyl)-4-hydroxyhydrocinnamamide) and of tris(2,4-di(tert-butyl)phenyl) phosphate, in particular that sold by CIBA under the name Irganox® B 1171, tetrakis(2,4-di(tert-butyl)phenyl) phosphite, in particular that sold by CIBA under the name Irgafos® P-EPQ, 2,4-bis(octylthiomethyl)-o-cresol, in particular that sold by CIBA under the name Irganox® 1520, 4,6-bis(dodecylthiomethyl)-o-cresol, in particular that sold by CIBA under the name Irganox® 1726.

The additional antioxidant(s) can be present in a composition according to the invention in a content ranging from 0.01% to 2.5% by weight, in particular from 0.05% to 1.5% by weight, more particularly from 0.1% to 0.5% by weight, with respect to the total weight of the composition.

In particular, a fragranced composition according to the invention comprises less than 0.5% by weight, preferably less than 0.1% by weight, more preferentially less than 0.05% by weight, indeed even less than 0.01% by weight, of additional anti-oxidizing agent(s). For example, a fragranced composition according to the invention is devoid of additional anti-oxidizing agent(s).

According to a specific embodiment, a composition according to the invention can also comprise butylated hydroxytoluene (BHT) as antioxidant.

According to a preferred embodiment, a composition according to the invention is preferably free from compounds liable to be harmful to human beings and/or the environment, that is to say that it comprises less than 0.2% by weight, in particular less than 0.1% by weight, preferably less than 0.05% by weight and more preferentially less than 0.01% by weight, relative to the total weight of the composition and even is devoid, of compounds liable to be harmful to human beings and/or the environment, in particular of butylated hydroxytoluene (BHT).

Thus, a composition according to the invention is in particular devoid of butylated hydroxytoluene (BHT).

### Additional Stabilizers

A fragranced composition according to the invention can also comprise one or more stabilizing agent(s) for the colour distinct from said sulfur-comprising antioxidant compound a).

Mention will be made, as stabilizers for the colour of fragranced compositions, of tris(tetramethylhydroxypiperidinol) citrate, such as the product sold under the name Tinoguard Q by Ciba-Geigy, Sodium Benzotriazolyl Butylphenol Sulfonate, such as the product sold under the name Tinoguard HS by Ciba-Geigy; Benzotriazolyl Dodecyl p-Cresol, such as the product sold under the name Tinoguard TL by Ciba-Geigy; Sodium Benzotriazolyl Butylphenol Sulfonate (and) Buteth-3 (and) Tributyl Citrate, such as the product sold under the trade name Cibafast H Liquid by Ciba-Geigy; Bumetrizole, such as the product sold under the name Tinoguard AS by Ciba-Geigy.

These stabilizers can be present in a content ranging from 0.005% to 2% by weight, with respect to the total weight of the composition, in particular from 0.01% to 0.5% by weight, with respect to the total weight of the composition.

In particular, a composition according to the invention comprises less than 0.1% by weight, in particular less than 0.05% by weight, of stabilising agent(s) for the colour, with respect to the total weight of the composition, indeed even is devoid of stabilizing agent for the colour.

### Vitamins

A fragranced composition according to the invention can also comprise vitamins and their derivatives, in particular their esters, such as niacinamide (3-pyridinecarboxamide), nicotinamide (vitamin B3), tocopherol (vitamin E) and its derivatives (such as tocopheryl acetate), ascorbic acid and its derivatives (vitamin C), retinol (vitamin A).

In particular, the vitamins or their derivatives can be chosen from tocopherol and its derivatives. The tocopherol can be found in one of its various forms, in particular the a, 3, y and 6 forms. Mention may be made, among tocopherol derivatives, of tocotrienols and esters.

Preferably, a composition according to the invention comprises at least one or more anti-oxidizing agent(s) chosen from tocopherol and tocopherol esters, in particular tocopherol acetate, more preferentially from α-tocopherol and α-tocopherol acetate.

In particular, a fragranced composition according to the invention comprises at least 0.001% by weight, in particular from 0.001% to 0.5% by weight, more particularly from 0.02% to 0.1% by weight, indeed even from 0.03% to 0.05% by weight, of tocopherol and/or its derivatives, with respect to the total weight of said fragranced composition.

### Solvent

In particular, a fragranced composition according to the invention is an aqueous composition, namely a composition comprising at least water as solvent(s), an alcoholic composition, namely a composition comprising at least one alcohol as solvent(s), or an aqueous/alcoholic composition, namely a composition comprising a mixture of water and of alcohol as solvents.

In particular, a fragranced composition according to the invention comprises at least 50% by weight, in particular from 60% to 95% by weight, more particularly from 70% to 90% by weight, of one or more solvent(s) chosen from water, an alcohol and their mixture, with respect to the total weight of said fragrancing composition.

Preferably, a fragranced composition according to the invention is an aqueous/alcoholic composition.

A fragranced composition according to the invention can comprise a mixture of water and of alcohol in an alcohol/water ratio by weight ranging from 50/50 to 99.5/0.5, preferably ranging from 70/30 to 99/1, especially ranging from 80/20 to 98/2, in particular ranging from 85/15 to 95/5.

As mentioned above, a fragranced composition according to the invention can be an alcoholic or aqueous/alcoholic composition.

The alcohols very particularly suitable for the invention are chosen from i) monoalcohols having from 2 to 6 carbon atoms, such as ethanol and isopropanol, and ii) glycols having from 2 to 8 carbon atoms, such as ethylene glycol, propylene glycol, 1,3-butylene glycol and dipropylene glycol.

Preferably, the alcohols are of natural origin. Mention may in particular be made of bioethanol.

In particular, a fragranced composition according to the invention comprises, as solvent(s), water, (bio)ethanol, pentylene glycol or a mixture of at least two of these compounds.

As mentioned above, a fragranced composition according to the invention can be an aqueous composition, namely a composition comprising water as solvent(s), or an aqueous/alcoholic composition, namely a composition comprising a mixture of water and of alcohol as solvents.

A fragranced composition according to the invention can comprise, besides water, at least one water-soluble solvent distinct from the alcohols defined above.

In the present invention, the term “water-soluble solvent” denotes a compound which is liquid at ambient temperature and water-miscible (miscibility in water of greater than 50% by weight at a temperature ranging from 20° C. to 25° C. and atmospheric pressure).

The water-soluble solvents which can be used in the composition of the invention can additionally be volatile. Mention may in particular be made, among the water-soluble solvents which can be used in the composition in accordance with the invention, of C₃ and C₄ ketones and C₂-C₄ aldehydes.

Alternatively, a fragranced composition according to the invention can be anhydrous, namely that it can comprise less than 2% by weight of water, in particular less than 1% by weight of water, more particularly less than 0.5% by weight of water, indeed even is completely devoid of water.

A fragranced composition according to the invention can also comprise at least one C₂-C₃₂ polyol. Within the meaning of the present invention, the term “polyol” should be understood as meaning any organic molecule comprising at least three free hydroxyl (—OH) groups.

Preferably, a polyol in accordance with the present invention is present in the liquid form at ambient temperature. A polyol suitable for the invention can be a compound of saturated or unsaturated and linear, branched or cyclic alkyl type carrying, on the alkyl chain, at least three —OH functions, in particular at least four —OH functions.

The polyols advantageously suitable for the formulation of a composition according to the present invention are those exhibiting in particular from 2 to 32 carbon atoms, preferably from 3 to 16 carbon atoms.

Preferably, the polyol can, for example, be chosen from pentaerythritol, trimethylolpropane, glycerol, polyglycerols, in particular glycerol oligomers, preferably diglycerol, and their mixtures.

### Additives

A fragranced composition according to the invention can additionally comprise any additive usually used in the field of fragrances, for instance emollients or softeners, in particular sweet almond oil, apricot kernel oil, sweeteners, moisturizing agents, in particular glycerol, soothing agents, in particular α-bisabolol, allantoin and Aloe vera; essential fatty acids, propellants, peptizing agents, fillers, cosolvents, surfactants, anti-foaming agents, polar additives, film-forming polymers, gelling agents, thickening agents, pH adjusters (acids or bases), neutralizing agents, chelating agents or preservatives, and their mixtures.

It is a matter of routine operations for a person skilled in the art to adjust the nature and the amount of the additives present in the compositions in accordance with the invention so that the cosmetic properties desired for the compositions are not thereby affected.

When they are present in the composition of the invention, these additives can be present in an amount ranging from 0.001% to 10% by weight and better still from 0.01% to 5% by weight, with respect to the total weight of the composition.

### d) Colorants

A fragranced composition according to the invention can also comprise at least one colorant. Generally, “colorant” is understood to denote any compound capable of colouring a composition, that is to say which absorbs in the visible spectrum, in particular so as to appear to the human eye to have a colour such as yellow, orange, red, purple, blue or green.

This (or these) colorant(s) is (or are) preferably chosen from particulate colorants, fat-soluble dyes, water-soluble dyes and their mixtures.

Within the meaning of the invention, the term “liposoluble dye” is understood to denote any natural or synthetic, generally organic, dye soluble in an oily phase or solvents miscible with a fatty substance.

The particulate colorants can be coloured pigments (i.e., other than the pigment UV screening agents), pearlescent agents and/or particles having metallic glints.

Pigments are naturally insoluble in the liquid hydrophilic and lipophilic phases usually employed in cosmetics. Alternatively, they can be rendered insoluble by formulation in lake form, if appropriate.

More particularly, the pigment has little or no solubility in aqueous/alcoholic media.

The pigments can be white or coloured, inorganic and/or organic, and coated or uncoated. Mention may be made, among the inorganic pigments, of metal oxides, in particular titanium dioxide, optionally surface-treated, zirconium oxide, zinc oxide or cerium oxide, and also iron oxide, titanium oxide or chromium oxide, manganese violet, ultramarine blue, chromium hydrate and ferric blue.

In particular, the colorant can be chosen from organic pigments and synthetic or natural water-soluble dyes, preferably from synthetic or natural water-soluble dyes.

Mention may be made, among the organic pigments of the invention, of carbon black, pigments of D & C type, in particular of azo, anthraquinone, nitro and/or triarylmethane form, and lakes based on cochineal carmine of barium, strontium, calcium and aluminium, preferably pigments of D & C type.

According to a specific embodiment of the invention, the colorant(s) of the invention is (are) chosen from water-soluble dyes. The water-soluble dyes can be anionic, cationic, zwitterionic or neutral, more particularly anionic (i.e. commonly referred to as “acidic” direct dyes for their affinity with alkaline substances).

The term “anionic dyes” is understood to mean any dye comprising, in its structure, at least one CO₂R′ or SO₃R′ substituent with R′ denoting a hydrogen atom or a cation originating from a metal or from an amine, or an ammonium ion.

The anionic dyes can be chosen from acid nitro direct dyes, acid azo dyes, acid azine dyes, acid triarylmethane dyes, acid indoamine dyes, acid anthraquinone dyes, indigoids and acid natural dyes.

Mention may be made, as anionic dyes suitable for the invention, of the dyes chosen from those of following formulae (G), (G′), (J), (J′), (K), (K′), (L) and (M) and their mixtures, in particular of formulae (G), (J) and (L) and their mixtures:
a) the anionic azo dyes of formulae (G) and (G′) below:

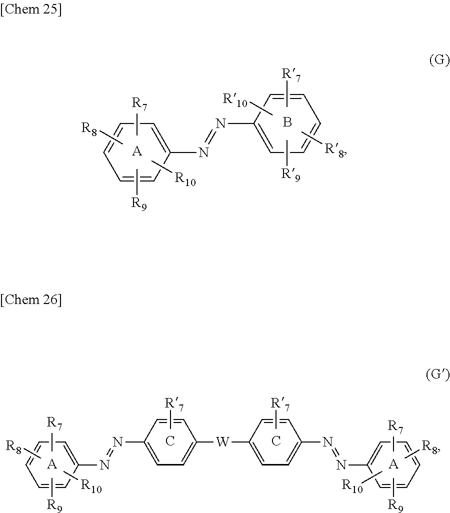

in which formulae R₇, R₈, R₉, R₁₀, R′₇, R′₈, R′₉ and R′₁₀, which are identical or different, represent a hydrogen or halogen atom or a group chosen from:
alkyl;
alkoxy, alkylthio;
hydroxyl, mercapto;
nitro, nitroso;
R^{o}—C(X)—X′—, R^{o}—X′—C(X)— or R^{o}—X′—C(X)—X″— with R^{o} representing a hydrogen atom or an alkyl or aryl group; X, X′ and X″, which are identical or different, representing an oxygen or sulfur atom or NR, with R representing a hydrogen atom or an alkyl group;
M⁺(O⁻)S(O)₂— with M⁺ representing a hydrogen atom or a cationic counterion;
M⁺(O⁻)CO— with M⁺ as defined above;
R″—S(O)₂— with R″ representing a hydrogen atom or an alkyl, aryl, (di)(alkyl)amino or aryl(alkyl)amino group; preferentially a phenylamino or phenyl group;
R″′—S(O)₂—X′— with R″′ representing an alkyl or aryl group which is optionally substituted, X′ being as defined above;
(di)(alkyl)amino;
aryl(alkyl)amino optionally substituted by one or more groups chosen from i) nitro; ii) nitroso; iii) M⁺(O⁻)S(O)₂— and iv) alkoxy with M⁺ as defined above;
optionally substituted heteroaryl; preferentially a benzothiazolyl group;
cycloalkyl; in particular cyclohexyl;
Ar—N═N— with Ar representing an optionally substituted aryl group; preferentially a phenyl optionally substituted by one or more alkyl, M⁺(O⁻)S(O)₂— or phenylamino groups;
or else two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted by one or more groups chosen from nitro; nitroso; M⁺(O⁻)S(O)₂—; hydroxyl; mercapto; (di)(alkyl)amino; R^{o}—C(X)—X′—; R^{o}—X′—C(X)—; R^{o}—X′—C(X)—X″—; Ar—N═N— and aryl(alkyl)amino which is optionally substituted; with M⁺, R^{o}, X, X′, X″ and Ar as defined above;
   and in which:
W represents a sigma a bond, an oxygen or sulfur atom, or a divalent —NR— radical with R as defined above or methylene —C(Ra)(Rb)— radical with Rₐ and Rb, which are identical or different, representing a hydrogen atom or an aryl group, or else Rₐ and Rb form, together with the carbon atom which carries them, a spiro cycloalkyl; preferentially, W represents a sulfur atom or Ra and Rb together form a cyclohexyl;
   it being understood that the formulae (G) and (G′) comprise at least one sulfonate M⁺(O⁻)S(O)₂— radical or one carboxylate M⁺(O⁻)CO— radical on one of the rings A, A′, B, B′ or C; preferentially sodium sulfonate.

Mention may be made, as examples of dyes of formula (G), of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment Red 57, Acid Red 68, Acid Red 73, Acid Red 135, Acid Red 138, Acid Red 184, Food Red 1, Food Red 13, Orange 4, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3; Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2; Food Yellow 3 or Sunset Yellow; and mention may be made, as examples of dyes of formula (G′), of: Acid Red 111, Acid Red 134, Acid Yellow 38;
b) the anthraquinone dyes of formulae (J) and (J′) below:

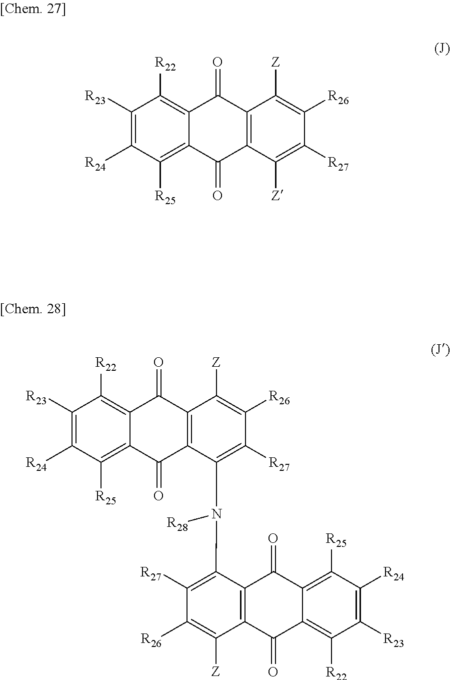

in which formulae (J) and (J′) R₂₂, R₂₃, R₂₄, R₂₅, R₂₆ and R₂₇, which are identical or different, represent a hydrogen or halogen atom or a group chosen from:
alkyl;
hydroxyl, mercapto;
alkoxy, alkylthio;
optionally substituted aryloxy or arylthio, preferentially substituted by one or more groups chosen from alkyl and M⁺(O⁻)S(O)₂— with M⁺ as defined above; aryl(alkyl)amino optionally substituted by one or more groups chosen from alkyl and M⁺(O⁻)S(O)₂— with M⁺ as defined above;
(di)(alkyl)amino
(di)(hydroxyalkyl)amino;
M⁺(O⁻)S(O)₂— with M⁺ as defined above;
and in which:
   Z′ represents a hydrogen atom or an NR₂₈R₂₉ group with R₂₈ and R₂₉, which are identical or different, representing a hydrogen atom or a group chosen from:
alkyl;
polyhydroxyalkyl, such as hydroxyethyl;
aryl, optionally substituted by one or more groups, particularly i) alkyl, such as methyl, n-dodecyl or n-butyl; ii) M⁺(O⁻)S(O)₂— with M⁺ as defined above; iii) R^{o}—C(X)—X′—, R^{o}—X′—C(X)— or R^{o}—X′—C(X)—X″— with R^{o}, X, X′ and X″ as defined above; preferentially, R^{o} represents an alkyl group;
cycloalkyl; in particular cyclohexyl;
   Z represents a group chosen from hydroxyl and NR′₂₈R′₂₉ with R′₂₈ and R′₂₉, which are identical or different, representing the same atoms or groups as R₂₈ and R₂₉ as defined above; it being understood that the formulae (J) and (J′) comprise at least one sulfonate M⁺(O⁻)S(O)₂— radical or one carboxylate M⁺(O⁻)CO— radical; preferentially sodium sulfonate.

Mention may be made, as examples of dyes of formula (J), of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT Violet No. 2; and mention may be made, as an example of a dye of formula (J′), of: Acid Black 48; d) the nitro dyes of formulae (K) and (K′) below:

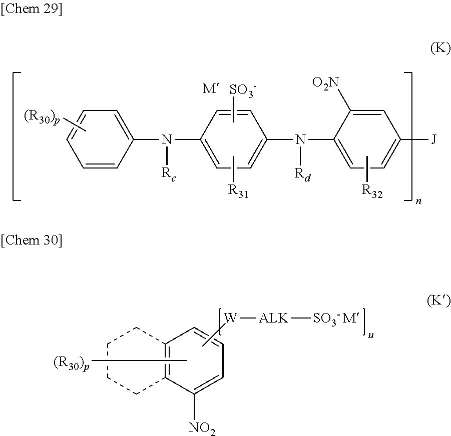

in which R₃₀, R₃₁ and R₃₂, which are identical or different, represent a hydrogen or halogen atom or a group chosen from:
alkyl;
alkoxy optionally substituted by one or more hydroxyl groups, alkylthio optionally substituted by one or more hydroxyl groups;
hydroxyl, mercapto;
nitro, nitroso;
polyhaloalkyl;
R^{o}—C(X)—X′—, R^{o}—X′—C(X)—, R^{o}—X′—C(X)—X″— with R^{o}, X, X′ and X″ as defined above;
M⁺(O⁻)S(O)₂— with M⁺ as defined above;
M⁺(O⁻)CO— with M⁺ as defined above;
(di)(alkyl)amino;
(di)(hydroxyalkyl)amino;
heterocycloalkyl, such as piperidino, piperazino or morpholino; in particular, R₃₀, R₃₁ and R₃₂ represent a hydrogen atom;
   and in which:
Rc and Rd, which are identical or different, represent a hydrogen atom or an alkyl group;
W is as defined above; W particularly represents an —NH— group;
ALK represents a linear or branched divalent C₁-C₆ alkylene group; in particular, ALK represents a —CH₂—CH₂— group;
n has the value 1 or 2;
p represents an integer of between 1 and 5 inclusive;
q represents an integer of between 1 and 4 inclusive;
u has the value 0 or 1;
when n has the value 1, J represents a nitro or nitroso group; particularly a nitro group;
when n has the value 2, J represents an oxygen or sulfur atom or a divalent —S(O)ₘ— radical with m representing an integer 1 or 2; preferentially, J represents an —SO₂— radical;
M′ represents a hydrogen atom or a cationic counterion;

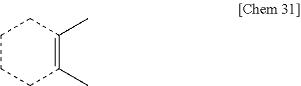

which is present or absent, represents a benzo group optionally substituted by one or more R₃₀ groups as defined above;
it being understood that the formulae (K) and (K′) comprise at least one sulfonate M⁺(O⁻)S(O)₂— radical or one carboxylate M⁺(O⁻)CO— radical; preferentially sodium sulfonate.

Mention may be made, as examples of dyes of formula (K), of: Acid Brown 13 and Acid Orange 3; mention may be made, as examples of dyes of formula (K′), of: Acid Yellow 1, the sodium salt of 2,4-dinitro-1-naphthol-7-sulfonic acid, 2-piperidino-5-nitrobenzenesulfonic acid, 2-(4′-N,N-(2″-hydroxyethyl)amino-2′-nitro)anilineethanesulfonic acid, 4-β-hydroxyethylamino-3-nitrobenzenesulfonic acid; EXT D&C Yellow 7;
e) the triarylmethane dyes of formula (L) below:

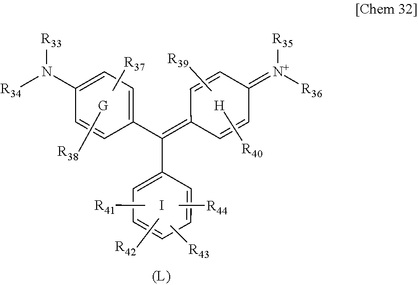

in which formula (L):
R₃₃, R₃₄, R₃₅ and R₃₆, which are identical or different, represent a hydrogen atom or a group chosen from alkyl, optionally substituted aryl and optionally substituted arylalkyl; particularly an alkyl and benzyl group optionally substituted by an M⁺(O⁻)S(O)ₘ— group with M⁺ and m as defined above;
R₃₇, R₃₈, R₃₉, R₄₀, R₄₁, R₄₂, R₄₃ and R₄₄, which are identical or different, represent a hydrogen atom or a group chosen from:
alkyl;
alkoxy, alkylthio;
(di)(alkyl)amino;
hydroxyl, mercapto;
nitro, nitroso;
R^{o}—C(X)—X′—, R^{o}—X′—C(X)— or R^{o}—X′—C(X)—X″— with R^{o} representing a hydrogen atom or an alkyl or aryl group; X, X′ and X″, which are identical or different, representing an oxygen or sulfur atom or NR, with R representing a hydrogen atom or an alkyl group;
M⁺(O⁻)S(O)₂— with M⁺ representing a hydrogen atom or a cationic counterion;
M⁺(O⁻)CO— with M⁺ as defined above;
or else two contiguous groups R₄₁ with R₄₂ or R₄₂ with R₄₃ or R₄₃ with R₄₄ together form a fused benzo group: I′; with I′ optionally substituted by one or more groups chosen from nitro; nitroso; M⁺(O⁻)S(O)₂—; hydroxyl; mercapto; (di)(alkyl)amino; R^{o}—C(X)—X′—; R^{o}—X′—C(X)— and R^{o}—X′—C(X)—X″—; with M⁺, R^{o}, X, X′ and X″ as defined above; in particular, R₃₇ to R₄₀ represent a hydrogen atom and R₄₁ to R₄₄, which are identical or different, represent a hydroxyl or M⁺(O⁻)S(O)₂— group; and when R₄₃ with R₄₄ together form a benzo group, it is preferentially substituted by an (O⁻)S(O)₂— group;
   it being understood that at least one of the rings G, H, I or I′ comprises at least one sulfonate (O⁻)S(O)₂— radical or one carboxylate (O⁻)CO— radical; preferentially sulfonate.

Mention may be made, as examples of dyes of formula (L), of: Acid Blue 1, Acid Blue 3, Acid Blue 7, Acid Blue 9, Acid Violet 49, Acid Green 3, Acid Green 5, Acid Green 50.
f) the xanthene-derived dyes of formula (M) below:

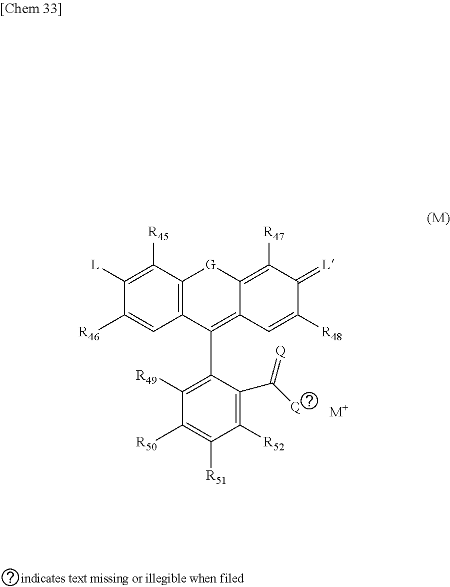

in which formula (M) R₄₅, R₄₆, R₄₇ and R₄₈, which are identical or different, represent a hydrogen atom or a halogen atom and R₄₉, R₅₀, R₅₁ and R₅₂, which are identical or different, represent a hydrogen or halogen atom or a group chosen from:
alkyl;
alkoxy, alkylthio;
hydroxyl, mercapto;
nitro, nitroso;
M⁺(O⁻)S(O)₂— with M⁺ representing a hydrogen atom or a cationic counterion;
M⁺(O⁻)CO— with M⁺ as defined above;
in particular, R₄₉, R₅₀, R₅₁ and R₅₂ represent a hydrogen or halogen atom; and in which:
G represents an oxygen or sulfur atom or an NRe group with Re representing a hydrogen atom or an alkyl group; in particular, G represents an oxygen atom;
L represents an alkoxide M⁺O⁻; a thioalkoxide M⁺S⁻ or an NRf group, with Rf representing a hydrogen atom or an alkyl group, and M⁺ as defined above; M⁺ is particularly sodium or potassium;
L′ represents an oxygen or sulfur atom or an ammonium group: N⁺RfRg, with Rf and Rg, which are identical or different, representing a hydrogen atom or an optionally substituted alkyl or aryl group; L′ particularly represents an oxygen atom or a phenylamino group optionally substituted by one or more alkyl or M⁺(O⁻)S(O)ₘ— groups with m and M⁺ as defined above;
Q and Q′, which are identical or different, represent an oxygen or sulfur atom; in particular, Q and Q′ represent an oxygen atom;
M⁺ is as defined above.

Mention may in particular be made, as colorants suitable for the invention, of synthetic or natural water-soluble dyes, such as, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 3, DC Green 5, FDC Blue 1 and their mixtures, more particularly chosen from FDC Red 4, DC Red 33, DC Orange 4, DC Yellow 5, DC Green 3, DC Green 5 and FDC Blue 1 and their mixtures.

Mention may be made, among the natural direct dyes which can be used according to the invention as colorant, of lawsone, juglone, alizarin, purpurin, carmine and carminic acid, kermesic acid, purpurogallin, protocatechualdehyde, indigo, isatin, curcumin, spinulosin, apigenidin, orceins, betanin (beetroot), chlorophyllin metal complexes, in particular chlorophyllin copper complex, methylene blue, caramel, sandalwood, Gardenia, Spirulina and riboflavin.

Within the meaning of the present invention, the term “pearlescent agents” is understood to denote coloured particles of any form, which are or are not iridescent, in particular produced by certain molluscs in their shells, or else synthesized, and which exhibit a colour effect via optical interference.

The pearlescent agents be chosen from pearlescent pigments, such as titanium oxide-coated mica covered with an iron oxide, titanium oxide-coated mica covered with bismuth oxychloride, titanium oxide-coated mica covered with chromium oxide, titanium oxide-coated mica covered with an organic dye and also pearlescent pigments based on bismuth oxychloride.

The pearlescent agents can more particularly have a yellow, pink, red, bronze, orangey, brown, gold and/or coppery colour or glint.

Within the meaning of the present invention, the term “particles having a metallic glint” means any compound, the nature, the size, the structure and the surface finish of which makes it possible for it to reflect the incident light, in particular in a non-iridescent manner.

The particles having a metallic glint which can be used in the invention can in particular be chosen from the particles of at least one metal and/or of at least one metal derivative, the particles comprising a single-material or multi-material organic or inorganic substrate, at least partially covered by at least one layer having a metallic glint comprising at least one metal and/or at least one metal derivative, and the mixtures of said particles. These particles are different from metal ions.

Mention may made, among the metals which can be present in said particles, for example, of Ag, Au, Cu, Al, Ni, Sn, Mg, Cr, Mo, Ti, Zr, Pt, Va, Rb, W, Zn, Ge, Te, Se and their mixtures or alloys. Ag, Au, Cu, Al, Zn, Ni, Mo, Cr and their mixtures or alloys (for example bronzes and brasses) are preferred metals.

The term “metal derivatives” denotes compounds derived from metals, in particular oxides, fluorides, chlorides and sulfides.

Preferably, the colorants, if present, are natural or of natural origin.

These colorants can be present in an amount ranging from 0.00005% to 0.005% by weight, with respect to the total weight of the composition, and in particular from 0.0001% to 0.001% by weight, with respect to the total weight of the composition.

### Composition

As mentioned above, a fragranced composition according to the invention comprises a) a least one sulfur-comprising antioxidant compound as defined above, b) at least one organic UV screening agent chosen from dibenzoylmethane derivatives as defined above and c) at least one fragrancing substance as described above.

Preferably, a composition according to the invention comprises:
a′) from 0.05% to 0.5%, in particular from 0.08% to 0.1%, of one or more sulfur-comprising antioxidant compound(s) chosen from dilauryl thiodipropionate, distearyl thiodipropionate, dilauryl dithiodipropionate, distearyl dithiodipropionate and their mixtures, in particular dilauryl thiodipropionate, with respect to the total weight of said fragranced composition;
b′) from 0.1% to 1%, in particular from 0.25% to 0.35%, of one or more organic UV screening agent(s) chosen from Butyl Methoxydibenzoylmethane and Isopropyl Dibenzoylmethane, in particular Butyl Methoxydibenzoylmethane, with respect to the total weight of said fragranced composition;
c′) from 1% to 35%, in particular from 10% to 25%, of one or more fragrancing substance(s), with respect to the total weight of said fragranced composition.

A fragranced composition according to the invention is preferably a cosmetic composition, namely a cosmetically acceptable composition, in order to be applied to keratin materials, in particular human keratin materials, without detrimentally affecting them.

It can be provided in any presentation form conventionally used for a topical application and in particular in the form of an aqueous, alcoholic or aqueous/alcoholic solution or suspension or of an oily solution or of a solution or dispersion of the lotion or serum type, of an emulsion of liquid or semi-liquid consistency of the milk type, which are obtained by dispersion of a fatty phase in an aqueous phase (O/W) or vice versa (W/O), or of a suspension or emulsion of soft consistency of (O/W) or (W/O) cream type, or of an aqueous or anhydrous gel, of an ointment, or of any other cosmetic form.

### Preparation of the Composition

A composition according to the invention can be obtained by any method known to a person skilled in the art for the formulation of coloured fragranced compositions.

In particular, a fragranced composition according to the invention can be obtained by simple mixing, at ambient temperature, namely at a temperature ranging from 20° C. to 25° C., of the compounds constituting the composition.

### Intended Use of the Composition

A composition according to the invention is a fragranced composition, generally suitable for topical application to the skin and thus generally comprising a physiologically acceptable medium.

Preferably, it is a cosmetic composition.

The invention applies not only to fragrancing products but also to cosmetic treatment or care products for keratin materials, in particular the skin, including the scalp, and the lips, containing an odorous substance.

A composition according to the invention can thus constitute a composition for cosmetic treatment, care or fragrancing of keratin materials and can in particular be provided in the form of an eau fraiche, eau de toilette, eau de parfum, aftershave lotion, eau de soin, silicone or hydrosilicone care oil or also eau de cologne. It can also be provided in the form of a fragranced two-phase lotion (eau de toilette phase/hydrocarbon oil and/or silicone oil phase), a body milk or a shampoo. According to a specific embodiment, a composition according to the invention can also be an elixir or a concrete.

Preferably, a composition according to the invention is provided in the form of an eau fraiche, eau de toilette, eau de parfum, eau de cologne or eau de soin, and more preferentially still it is an eau de parfum.

A composition according to the invention can also constitute any form of cosmetic composition for caring for and/or for making up keratin materials.

The compositions according to the invention can be packaged in the form of bottles.

A fragrancing composition according to the invention can be diffused according to various systems, such as sprays, aerosols or piezoelectric devices.

They can also be applied in the form of fine particles by means of mechanical or propellant gas pressurization devices. The devices in accordance with the invention are well known to a person skilled in the art and comprise pump-action sprays, aerosol containers comprising a propellant and also aerosol pumps using compressed air as propellant. The latter are in particular described in U.S. Pat. Nos. 4,077,441 and 4,850,517.

The compositions packaged as aerosols in accordance with the invention generally contain conventional propellants, such as, for example, dimethyl ether, isobutane, n-butane or propane.

Preferably, a composition according to the invention is diffused in spray form.

The invention is illustrated in greater detail by the examples presented below. Unless otherwise indicated, the amounts indicated are expressed as percentages by weight.

### EXAMPLES

### Measurement and Evaluation Methods

The stability of the compositions is evaluated by observation, visually, of the change in the colour over time and by olfactory evaluation of the change in the notes of the fragranced composition.

In particular, the compositions are poured into 50 ml bottles pots made of borosilicate glass and are then observed after storage for 2 months, at atmospheric pressure and under various conditions:
a) in the refrigerator, at a temperature of 4° C.;
b) at ambient temperature, namely of between 20° C. and 25° C., with the exclusion of light;
c) in an oven, at a temperature of 37° C.;
d) in an oven, at a temperature of 45° C.;
e) in natural light (orientation north, location Paris) and at ambient temperature, namely of between 20° C. and 25° C., then:
e.1) 16 hours in a SunTest CPS+ ageing device of Atlas trade name, equipped with a xenon lamp, an irradiating light source, the spectral distribution of which is close to that of the sun, delivering an energy of 765 W/m², simulating exposure to neon lights in a shop; and
e.2) 2 weeks in an oven at a temperature of 55° C., simulating an extreme condition for the fragrance.

Storage of the compositions for 2 months at 45° C. simulates an accelerated ageing of the product corresponding to a shelf life of 3 years.

### Starting Materials

The compositions of the examples which follow employ different fragrances, named A to I, belonging to different olfactory families, as described in detail in the tables.

### Example 1

### Preparation of Fragranced Compositions

Different fragranced compositions A, B and C according to the invention and fragranced compositions a, b and c outside the invention are prepared using the proportions by weight as described in detail in Table 2 below.

The values are expressed as percentages by weight, with respect to the total weight of the fragranced composition.

TABLE 2

| | Test A | Control a | Test B | Control b | Test C | Control c |
|---|---|---|---|---|---|---|
| Fragrance(*)(INCI name: | Fragrance | Fragrance | Fragrance | Fragrance | Fragrance | Fragrance |
| Fragrance) | A 12 | A 12 | B 18 | B 18 | C 17.5 | C 17.5 |
| Non-denatured alcohol | 78.1 | 78.1 | 79.0 | 79.0 | 78.4 | 78.4 |
| Butyl | 0.3 | — | 0.3 | — | 0.3 | 0.5 |
| Methoxydibenzoylmethane | | | | | | |
| Parsol 1789 | | | | | | |
| Ethylhexyl salicylate | — | — | — | — | — | 0.5 |
| Neo Heliopan OS | | | | | | |
| Pentaerythrityl Tetra-Di-T- | | | | | — | 0.1 |
| Butyl | | | | | | |
| Hydroxyhydrocinnamate | | | | | | |
| Tinoguard TT | | | | | | |
| Dilauryl thiodipropionate | 0.1 | — | 0.1 | — | 0.1 | — |
| Tinogard DA | | | | | | |
| Dye (at 0.1% in 70° | 0.3 | 0.3 | — | — | 0.096 | 0.096 |
| alcohol)(**) | | | | | | |
| Dye (at 0.3% in 70° | — | — | — | — | 0.070 | 0.070 |
| alcohol)(***) | | | | | | |
| Microbiologically clean | q.s. | q.s. | q.s. | q.s. | q.s. | q.s. |
| deionized water (INCI | for 100 | for 100 | for 100 | for 100 | for 100 | for 100 |
| name: Water/Aqua) | | | | | | |

(*)Fragrance A, characterized by: Top: Hesperidic (bergamot, mandarin, lemon) (Cologne effect), Aromatic (sage, basil, rosemary, lavender), Fruity (cucumber, melon); Middle: Floral (geranium, hedione, DHM); Base: Marine (ambergris, calone), Woody (cedar), Musky (galaxolide); Fragrance B, characterized by: Top: Hesperidic (bergamot), Spicy (ginger), Green Fruity (apple); Middle: Floral (lily, carnation, freesia, violet), Aromatic (tea); Base: Musky (galaxolide), Wet Woody (oakmoss, patchouli), Woody (sandalwood); Fragrance C, characterized by; Top: Green Fruity (apple, pear), Green (lawn), Hesperidic (bergamot); Middle: Floral (magnolia, rose, jasmine, orange blossom), Tea (Earl Grey); Base: Gourmand (vanilla, caramel, pyrazine, popcorn), Woody (patchouli, cedar), Musky (galaxolide).
(**)Test A and Control a: mixture of Blue 1 and Red 33; Test C and Control c: Ext. Violet 2
(***)Test C and Control c: mixture of Yellow 5 and Red 33

The tests A and B are, on average, over all the tests described in the section Measurement and evaluation methods, more stable olfactorily and in colour, respectively, than the comparative tests a and b, devoid of Butyl Methoxydibenzoylmethane and of dilauryl thiodipropionate.

Furthermore, the composition C according to the invention is at least as stable olfactorily and in colour, indeed even more stable olfactorily and in colour, than the composition c comprising ethylhexyl salicylate in combination with Butyl Methoxydibenzoylmethane and Pentaerythrityl Tetra-Di-T-Butyl Hydroxyhydrocinnamate.

### Example 2

### Preparation of Fragranced Compositions

Different fragranced compositions D, E, F and G according to the invention and fragranced compositions d, e, f and g outside the invention are prepared using the proportions by weight as described in detail in Table 3 below.

The values are expressed as percentages by weight, with respect to the total weight of the fragranced composition.

TABLE 3

| | | Control | | Control | | Control | | Control |
|---|---|---|---|---|---|---|---|---|
| | Test D | d | Test E | e | Test F | f | Test G | g |
| Fragrance(*)(INCI name: | Fragrance | Fragrance | Fragrance | Fragrance | Fragrance | Fragrance | Fragrance | Fragrance |
| Fragrance) | D 19.9 | D 19.9 | E 17 | E 17 | F 17 | F 17 | G 20 | G 20 |
| Methyl Anthranilate | — | — | — | — | — | — | 0.0045 | 0.0045 |
| Non-denatured alcohol | 77.7 | 77.7 | 77.2 | 77.2 | 77 | 77 | 65 | 65 |
| Butyl | 0.3 | 0.2 | 0.3 | 0.2 | 0.3 | 0.2 | 0.3 | 0.2 |
| Methoxydibenzoylmethane | | | | | | | | |
| Parsol 1789 | | | | | | | | |
| Ethylhexyl salicylate | — | 0.2 | — | 0.2 | — | 0.1 | — | — |
| Neo Heliopan OS | | | | | | | | |
| Diethylamino | — | — | — | — | — | 0.05 | — | — |
| Hydroxybenzoyl Hexyl | | | | | | | | |
| Benzoate | | | | | | | | |
| Tris(tetramethylhydroxypiperidinol) | — | — | — | 0.03 | — | 0.03 | — | — |
| citrate | | | | | | | | |
| Tinogard Q | | | | | | | | |
| Dilauryl thiodipropionate | 0.1 | — | 0.1 | — | 0.1 | — | 0.1 | — |
| Tinogard DA | | | | | | | | |
| Dye (at 0.1% in 70° | — | — | — | — | 0.076 | 0.076 | 0.83 | 0.83 |
| alcohol)(**) | | | | | | | | |
| Dye (at 0.3% in 70° | 0.104 | 0.104 | 0.044 | 10.044 | 0.18 | 0.18 | — | — |
| alcohol)(***) | | | | | | | | |
| Microbiologically clean | q.s. | q.s. | q.s. | q.s. | q.s. | q.s. | for 100 | q.s. |
| deionized water (INCI | for 100 | for 100 | for 100 | for 100 | for 100 | for 100 | q.s. | for 100 |
| name: Water/Aqua) | | | | | | | | |

(*)Fragrance D, characterized by: Top: Hesperidic (bergamot, citron), Spicy (pink peppercorn), Aromatic (mint); Middle: Fruity (watermelon, melon, peach), Floral (peony, jasmine); Base: Woody (cedar), Amber (ciste labdanum), Musky (galaxolide); Fragrance E, characterized by: Top: Hesperidic (bergamot, orange, mandarin), Green (galbanum), Fruity (pear, apple); Middle: Floral (lily of the valley, peony, hyacinth, rose), Fruity (pineapple, strawberry); Base: Gourmand (vanilla, cocoa, caramel), Woody (cedar), Musky (galaxolide); Fragrance F, characterized by: Top: Hesperidic (bergamot, lemon, grapefruit), Aromatic (DHM, lavender, thyme, rosemary), ozone note (marine, iodized); Middle: Floral (hedione, geranium); Base: Woody (cedar, cashmeran), Musky (galaxolide); Fragrance G, characterized by: Top: Hesperidic (sweet orange, mandarin, bergamot); Middle: Floral (jasmine, orange blossom), Fruity (pear, blackcurrant); Base: Gourmand (vanilla, roast coffee), Woody (cedar, patchouli), Musky (galaxolide).
(**)Test F and Control f: Ext. Violet 2; Test G and Control g: mixture of Yellow 6 and Red 4
(***)Test D and Control d: mixture of Green 5 and Red 33; Test E and Control e: mixture of Yellow 5, Red 4 and Red 33; Test F and Control f: mixture of Orange 4, Red 4 and Blue 1

The composition D according to the invention is at least as stable olfactorily, on average, over all the tests described in the section Measurement and evaluation methods, indeed even more stable olfactorily, than the composition d comprising ethylhexyl salicylate in combination with Butyl Methoxydibenzoylmethane.

The composition E according to the invention is at least as stable olfactorily, on average, over all the tests described in the section Measurement and evaluation methods, indeed even more stable olfactorily, than the composition e comprising ethylhexyl salicylate and tris(tetramethylhydroxypiperidinol) citrate in combination with Butyl Methoxydibenzoylmethane.

With regard to the composition F according to the invention, it is at least as stable olfactorily, on average, over all the tests described in the section Measurement and evaluation methods, indeed even more stable olfactorily, than the composition f comprising ethylhexyl salicylate, tris(tetramethylhydroxypiperidinol)citrate and Diethylamino Hydroxybenzoyl Hexyl Benzoate in combination with Butyl Methoxydibenzoylmethane.

The test G is, on average, over all the tests described in the section Measurement and evaluation methods, more stable olfactorily than the comparative test g, devoid of dilauryl thiodipropionate.

### Example 3

### Preparation of Fragranced Compositions

Different fragranced compositions H and H′ according to the invention and fragranced compositions h and h′ outside the invention are prepared using the proportions by weight as described in detail in Table 4 below.

The values are expressed as percentages by weight, with respect to the total weight of the fragranced composition.

TABLE 4

| | Test H | Control h | Test H′ | Control h′ |
|---|---|---|---|---|
| Fragrance(*)(INCI name: | Fragrance | Fragrance | Fragrance | Fragrance |
| Fragrance) | H 23 | H 23 | H 23 | H 23 |
| Non-denatured alcohol | 69 | 69 | 69 | 69 |
| α-Tocopherol | — | — | 0.036 | 0.036 |
| Butyl Methoxydibenzoyl- | 0.3 | — | 0.3 | — |
| methane Parsol 1789 | | | | |
| Benzotriazolyl Dodecyl | — | 0.49 | — | 0.49 |
| p-Cresol Tinogard TL | | | | |
| Tris(tetramethylhydroxy- | — | 0.03 | — | 0.03 |
| piperidinol) citrate | | | | |
| Tinogard Q | | | | |
| Dilauryl thiodipropionate | 0.1 | — | 0.1 | — |
| Tinogard DA | | | | |
| Ext. Violet 2 at 0.1% in | 0.375 | 0.375 | 0.375 | 0.375 |
| 70° alcohol | | | | |
| Red 33 at 0.3% in 70° | 0.045 | 0.045 | 0.045 | 0.045 |
| alcohol | | | | |
| Microbiologically clean | q.s. | q.s. | q.s. | q.s. |
| deionized water (INCI | for 100 | for 100 | for 100 | for 100 |
| name: Water/Aqua) | | | | |

(*)Fragrance H, characterized by:
Top: Hesperidic (bergamot, sweet orange), Spicy (pink peppercorn, nutmeg), Green Fruity (pear); Middle: Red Fruits Fruity (cherry, raspberry, blackberry, redcurrant), Rose Floral (rose, peony), Violet Floral (ionones), Jasmine Floral (jasmine); Base: Balsamic (vanilla, benzoin), Coumarin (tonka bean), Woody (sandalwood).

The tests H and H′ are, on average, over all the tests described in the section Measurement and evaluation methods, more stable olfactorily respectively, than the comparative tests h et h′ comprising Benzotriazolyl Dodecyl p-Cresol and tris(tetramethylhydroxypiperidinol) citrate in combination with Butyl Methoxydibenzoylmethane.

Furthermore, the composition H′ according to the invention, additionally comprising α-tocopherol, is more stable olfactorily, on average, over all the tests described in the section Measurement and evaluation methods, than the composition H according to the invention devoid of α-tocopherol.

### Example 4

### Preparation of Fragranced Compositions

Different fragranced compositions 1 to 6 according to the invention and fragranced compositions 7 and 8 outside the invention are prepared using the proportions by weight as described in detail in Table 5 below.

The values are expressed as percentages by weight, with respect to the total weight of the fragranced composition.

TABLE 5

| | | | | | | | Control | Control |
|---|---|---|---|---|---|---|---|---|
| | Test 1 | Test 2 | Test 3 | Test 4 | Test 5 | Test 6 | 7 | 8 |
| Fragrance I(*)(INCI name: | 25.00 | 25.00 | 25.00 | 25.00 | 25.00 | 25.00 | 25.00 | 25.00 |
| Fragrance) | | | | | | | | |
| Methyl Anthranilate | 0.0049 | 0.0049 | 0.0049 | 0.0049 | 0.0049 | 0.0049 | 0.0049 | 0.0049 |
| Non-denatured alcohol | 68.72 | 68.72 | 68.72 | 68.72 | 68.72 | 68.72 | 68.72 | 68.72 |
| Butyl | 0.375 | 0.250 | 0.500 | 0.125 | 0.375 | 0.250 | 0.250 | — |
| Methoxydibenzoylmethane | | | | | | | | |
| Parsol 1789 | | | | | | | | |
| Dilauryl thiodipropionate | 0.025 | 0.050 | 0.050 | 0.075 | 0.075 | 0.100 | — | 0.050 |
| Tinogard DA | | | | | | | | |
| Red 4 at 0.3% in 70° | 0.123 | 0.123 | 0.123 | 0.123 | 0.123 | 0.123 | 0.123 | 0.123 |
| alcohol | | | | | | | | |
| Red 33 at 0.3% in | 70°0.005 | 0.005 | 0.005 | 0.005 | 0.005 | 0.005 | 0.005 | 0.005 |
| alcohol | | | | | | | | |
| Microbiologically clean | q.s. | q.s. | q.s. | q.s. | q.s. | q.s. | q.s. | q.s. |
| deionized water (INCI | for 100 | for 100 | for 100 | for 100 | for 100 | for 100 | for 100 | for 100 |
| name: Water/Aqua) | | | | | | | | |

(*) Fragrance I, characterized by: Top: Fruity (blackcurrant, pear), Hesperidic (bergamot, orange); Middle: Powdery (iris, ionones), White Flowers (jasmine, orange blossom); Base: Gourmand (sugared almond, vanilla, tonka), Woody (patchouli).

As described in detail in the section Measurement and evaluation methods, the compositions of the tests 1 to 8 are poured into 50 ml bottles pots made of borosilicate glass and are then evaluated after storage for 2 months, at atmospheric pressure and under the condition e).

The olfactory evaluation is carried out by a panel of 7 evaluators by comparison with the compositions having a composition stored at 4° C. The odour is graded from 0 to 10, a grade equal to 10 being given for an odour identical to that of the composition stored at 4° C. Thus, a higher grade is associated with a better olfactory stability. The averages obtained are given in Table 6.

TABLE 6

| | | | | | | | Control | Control |
|---|---|---|---|---|---|---|---|---|
| | Test 1 | Test 2 | Test 3 | Test 4 | Test 5 | Test 6 | 7 | 8 |
| Odour | 5.0 | 5.6 | 5.6 | 5.7 | 6.3 | 7.1 | 4.6 | 3.1 |
| (averaged | | | | | | | | |
| results) | | | | | | | | |

The olfactory stability of the compositions 1 to 6 according to the invention is improved in comparison with the control tests 7 and 8 respectively devoid of dilauryl thiodipropionate and of Butyl Methoxydibenzoylmethane. Furthermore, an excellent olfactory stability is obtained for the compositions according to the invention, in particular for the compositions of tests 5 and 6.

1-16. (canceled)

17. A composition comprising:
a) at least one sulfur-containing antioxidant compound chosen from compounds of formulae (I) or (II), optical and geometrical isomers thereof, salts thereof, solvates thereof, or mixtures of two or more thereof: 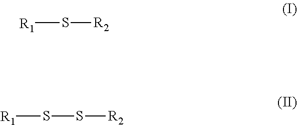
wherein in formulae (I) and (II):
R₁ and R₂ are independently chosen from a linear or branched (C₁-C₂₄)alkyl radical, a linear or branched (C₂-C₁₈)alkenyl radical, a (C₅-C₁₂)cycloalkyl radical, a phenylalkyl radical comprising from 7 to 15 carbon atoms or a phenyl radical,
the alkyl radical and/or the alkenyl radical are optionally substituted by one or more group(s) chosen from —OH, —O—C(O)—R₃, —C(O)—O—R₃, —C(O)—R₃, —OR′₃ and —NH₂ groups, and/or
the alkyl radical and/or the alkenyl radical are optionally interrupted by one or more heteroatoms,
the phenyl radical and/or the phenylalkyl radical are optionally substituted on the phenyl by 1 to 3 linear or branched (C₁-C₁₀)alkyl radicals,
R₃ is independently chosen from a hydrogen atom, a linear or branched (C₁-C₁₈)alkyl radical, a (C₅-C₁₂)cycloalkyl radical, a linear or branched (C₃-C₈)alkenyl radical, a (C₆-C₁₄)aryl radical or a (C₇-C₁₅)aralkyl radical, and
R′₃ represents a linear or branched (C₁-C₁₈)alkyl radical;
b) at least one organic UV screening agent chosen from dibenzoylmethane derivatives; and
c) at least one fragrancing substance.

18. The composition of claim 17, wherein the at least one sulfur-containing antioxidant compound chosen from compounds of formulae (I) or (II), optical and geometrical isomers thereof, salts thereof, solvates thereof, or mixtures of two or more thereof a) is chosen from thiodipropanoic acid, dithiodipropanoic acid, thiodipropanoic acid derivatives, dithiodipropanoic acid derivatives, or mixtures of two or more thereof.

19. The composition of claim 17, wherein the at least one sulfur-containing antioxidant compound chosen from compounds of formulae (I) or (II), optical and geometrical isomers thereof, salts thereof, solvates thereof, or mixtures of two or more thereof a) is chosen from dilauryl thiodipropionate, distearyl thiodipropionate, dilauryl dithiodipropionate, distearyl dithiodipropionate, or mixtures of two or more thereof.

20. The composition of claim 17, wherein the total amount of sulfur-containing antioxidant compound(s) chosen from compounds of formulae (I) or (II), optical and geometrical isomers thereof, salts thereof, solvates thereof, or mixtures of two or more thereof a) ranges from 0.01% to 5% by weight, relative the total weight of the composition.

21. The composition of claim 17, wherein the at least one organic UV screening agent b) is chosen from butyl methoxydibenzoylmethane, isopropyl dibenzoylmethane, or a mixture thereof.

22. The composition of claim 17, wherein the total amount of organic UV screening agent(s) b) ranges from 0.01% to 5% by weight, relative to the total weight of the composition.

23. The composition of claim 17, wherein the at least one fragrancing substance(s) c) is chosen from essential oils, fragrances and aromas of synthetic or natural origin, or mixtures thereof.

24. The composition of claim 17, wherein the total amount of fragrancing substance(s) c) is present in an amount of at least 0.5% by weight, relative to the total weight of the composition.

25. The composition of claim 17, further comprising d) at least one vitamin.

26. The composition of claim 25, wherein the at least one vitamin is chosen from tocopherol, tocopherol esters, or mixtures thereof.

27. The composition of claim 25, wherein the at least one vitamin is tocopherol acetate.

28. The composition of claim 17, wherein the composition is aqueous, alcoholic, or aqueous/alcoholic.

29. The composition of claim 17, further comprising e) at least one solvent chosen from water, alcohols, or mixtures thereof.

30. The composition of claim 29, wherein the total amount of solvent(s) e) is at least 50% by weight, relative to the total weight of the composition.

31. The composition of claim 17, further comprising f) at least one colorant.

32. The composition of claim 31, wherein the at least one colorant f) is chosen from organic pigments.

33. The composition of claim 17, further comprising g) at least one additional anti-oxidizing agent different from the at least one sulfur-containing antioxidant compound a).

34. The composition of claim 33, wherein the g) at least one additional anti-oxidizing agent is chosen from butylated hydroxytoluene, and is present in an amount of less than 0.2% by weight, relative to the total weight of the composition.

35. The composition of claim 17, wherein when the sulfur-containing antioxidant compound a) is a stabilizer, the composition further comprises h) at least one additional stabilizing agent different from the sulfur-containing antioxidant compound a).

36. A method for treating keratin materials, comprising applying to the keratin materials a composition comprising:
a) at least one sulfur-containing antioxidant compound chosen from compounds of formulae (I) or (II), optical and geometrical isomers thereof, salts thereof, solvates thereof, or mixtures of two or more thereof: 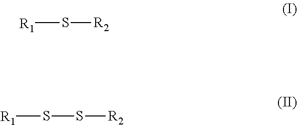
wherein in formulae (I) and (II):
R₁ and R₂ are independently chosen from a linear or branched (C₁-C₂₄)alkyl radical, a linear or branched (C₂-C₁₈)alkenyl radical, a (C₅-C₁₂)cycloalkyl radical, a phenylalkyl radical comprising from 7 to 15 carbon atoms or a phenyl radical,
the alkyl radical and/or the alkenyl radical are optionally substituted by one or more group(s) chosen from —OH, —O—C(O)—R₃, —C(O)—O—R₃, —C(O)—R₃, —OR′₃ and —NH₂ groups, and/or
the alkyl radical and/or the alkenyl radical are optionally interrupted by one or more heteroatoms,
the phenyl radical and/or the phenylalkyl radical are optionally substituted on the phenyl by 1 to 3 linear or branched (C₁-C₁₀)alkyl radicals,
R₃ is independently chosen from a hydrogen atom, a linear or branched (C₁-C₁₈)alkyl radical, a (C₅-C₁₂)cycloalkyl radical, a linear or branched (C₃-C₈)alkenyl radical, a (C₆-C₁₄)aryl radical or a (C₇-C₁₅)aralkyl radical, and
R′₃ represents a linear or branched (C₁-C₁₈)alkyl radical;
b) at least one organic UV screening agent chosen from dibenzoylmethane derivatives; and
c) at least one fragrancing substance.
